(19) United States
(12) Patent Application Publication
(10) Pub. No.: US 2008/0070961 A1
(43) Pub. Date: Mar. 20, 2008

(54) Polymorphs of N-(2-acetyl-4,6-dimethylphenyl)-3-{[(3,4 dimethyl-5-isoxazolyl)-amino]sulfonyl}-2-thiophene-carboxamide

(76) Inventors: John F. Reichwein, Basking Ridge, NJ (US); David T. Jonaitis, Brookston, IN (US); Mark C. Andres, Lafayette, IN (US)

Correspondence Address: JONES DAY, 222 EAST 41ST ST, NEW YORK, NY, 10017, US

(21) Appl. No.: 11/890,217

(22) Filed: Aug. 3, 2007

Related U.S. Application Data
(60) Provisional application No. 60/835,781, filed on Aug. 4, 2006.

(51) Int. Cl. A61K 31/42; A61P 11/00; A61P 25/00; A61P 27/02; A61P 29/00; A61P 9/00; C07D 261/14

(52) U.S. Cl. 514/380; 548/246

(57) ABSTRACT

N-(2-acetyl-4,6-dimethylphenyl)-3-([(3,4 dimethyl-5-isoxazolyl)aminosulfonyl}-2-thiophenecarboxamide, is provided here in the form of three polymorphs (Forms A, C and E). Forms A, C and E are specified by their peaks in their X-ray powder diffraction patterns, their absorption peaks in their infrared absorption spectra in potassium bromide, their peaks in their Raman absorption spectra, or their melting points.

### RELATED APPLICATION

Priority is claimed herein under 35 U.S.C. §119(e) to U.S. Provisional Patent Application No. 60/835,781, filed Aug. 4, 2006, entitled “POLYMORPHS OF N-(2-ACETYL-4,6-DIMETHYLPHENYL)-3-{[(3,4DIMETHYL-5-ISOXAZOLYL)-AMINO]SULFONYL}-2-THIOPHENE-CARBOXAMIDE.” The disclosure of the above-referenced application is incorporated by reference herein in its entirety.

### FIELD

Provided herein are polymorphs of N-(2-acetyl-4,6-dimethylphenyl)-3-{[(3,4dimethyl-5-isoxazolyl)-amino]sulfonyl}-2-thiophenecarboxamide and processes for producing them.

### BACKGROUND

N-(2-acetyl-4,6-dimethylphenyl)-3-{[(3,4dimethyl-5-isoxazolyl)-amino]sulfonyl}-2-thiophenecarboxamide modulates the activity of the endothelin family of peptides and is useful for the treatment of endothelin-mediated disorders. The compound's use as a pharmaceutical product may require storage for an extended period of time. Thus, the stability of this compound (bulk pharmaceutical chemicals) against heat and humidity during the storage period is very important. Therefore, a more stable form of this compound is desired.

### SUMMARY

It has been found that polymorphs of N-(2-acetyl-4,6-dimethylphenyl)-3-{[(3,4dimethyl-5-isoxazolyl)-amino]sulfonyl}-2-thiophenecarboxamide, Forms A, C and E and an amorphous form, can be selectively produced on an industrial scale by crystallization of this compound from appropriate solvents and conditions. Further, it has been found that these species of polymorphs can be interconverted to Form A under suitable conditions.

In particular, three polymorphs of N-(2-acetyl-4,6-dimethylphenyl)-3-{[(3,4dimethyl-5-isoxazolyl)-amino]sulfonyl}-2-thiophenecarboxamide, Forms A, C and E and an amorphous form, having the chemical structure: 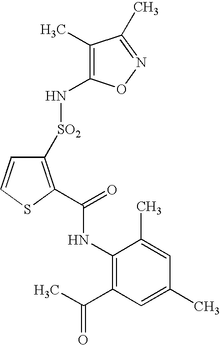
can be selectively produced and are distinguishable based upon the characteristic peaks in their X-ray powder diffraction (XRPD) patterns, infrared absorption spectra, Raman spectra and melting points.
Methods and Conditions of the Measurement of XRPD Patterns
Method of the Measurement

The XRPD analysis was measured on a Shimadzu XRD-6000 X-ray powder diffractometer on the samples by the following conditions.

Condition of the Measurement

| | |
|---|---|
| Target | Cu KΨ |
| Filter | monochro |
| Voltage | 40 kV |
| Current | 40 mA |
| Slit IDS | RS 0.15 nm SS 1° |
| Scan speed | 3°/min |
| Range | 2.5 to 40 |

Method and Condition of the Measurement of Infrared Absorption

The infrared absorption spectra in potassium bromide were measured on a Nicolet model 860 Fourier transform infrared (FT-IR) spectrophotometer.

Method and Condition of the Measurement of Raman Absorption

The Raman spectra were acquired on a Raman bench interfaced to a Nicolet Magna 860 FT-IR spectrophotometer.

Polymorph A (Form A)

The major peaks in the XRPD pattern of Form A expressed in degrees 2-theta are at approximately 11.26, 15.34, 16.06, 19.32, 22.32, 22.9, 24.56, 25.02, 26.34 and 28.68

FIGS. 1-5 show the XRPD pattern of Form A.

The peaks (cm⁻¹) in the infrared absorption spectra in potassium bromide of Form A are: 3810, 3156 (broad), 1466, 1396, 1363, 1135, 999, 908, 902 and 850.

FIG. 6 shows the infrared absorption spectra in potassium bromide of Form A.

The peaks (cm⁻¹) in the Raman spectra of Form A are: 3100, 2970, 1414, 1350, 850 and 640.

FIG. 7 shows the Raman spectra of Form A.

Based on the characterization data, Form A appears to be an unsolvated, non-hygroscopic, crystalline material that melts at 144° C.

Polymorph C (Form C)

The major peaks in the XRPD pattern of Form A expressed in degrees 2-theta are at approximately 7.56, 14.54, 15.96, 16.4, 19.04, 21.24 and 25.74.

FIGS. 1 and 11 shows the XRPD pattern of Form C.

The peaks (cm⁻¹) in the infrared absorption spectra of Form C in potassium bromide are: 3502, 3241(broad), 1684, 1525, 1402, 1293, 1140, 1017, 927(broad), 916, 896, 873, 784, 775, 746, 728, 706, 680, 653, 580 and 513.

FIG. 12 shows the infrared absorption spectra in potassium bromide of Form C.

The peaks (cm⁻¹) in the Raman spectra of Form Care: 3083, 1684, 1291, 1221, 1179 and 867.

FIG. 13 shows the Raman spectra of Form C.

The melting point of polymorph C is 143° C.

Polymorph E (Form E)

The major peaks in the XRPD pattern of Form A expressed in degrees 2-theta are at approximately 10.54, 14.66, 16.2, 20.04, 22.44, 23.82 and 24.82.

FIGS. 1 and 17 show the XRPD pattern of Form E.

The peaks (cm⁻¹) in the infrared absorption spectra of Form E in potassium bromide are: 3271(broad), 3129, 3005, 2943(broad), 1521, 1183, 1169, 1072, 1042, 911, 855, 752 and 645.

FIG. 18 shows the infrared absorption spectra in potassium bromide of Form E.

The peaks (cm⁻¹) in the Raman spectra of Form E are: 3131, 1418, 1066 and 645.

FIG. 19 shows the Raman spectra of Form E.

The melting point of polymorph E is 149° C.

### BRIEF DESCRIPTION OF THE DRAWINGS

FIG. 1 is the XRPD pattern of the polymorphs A, C, E and amorphous form.

FIG. 2 is the XRPD pattern of the polymorph A, lot 1.

FIG. 3 is the XRPD pattern of the polymorph A, lot 2.

FIG. 4 is the XRPD pattern of the polymorph A, lot 3.

FIG. 5 is the XRPD pattern of the polymorph A, lot 4.

FIG. 6 is the TG/IR absorption spectra of the polymorph A.

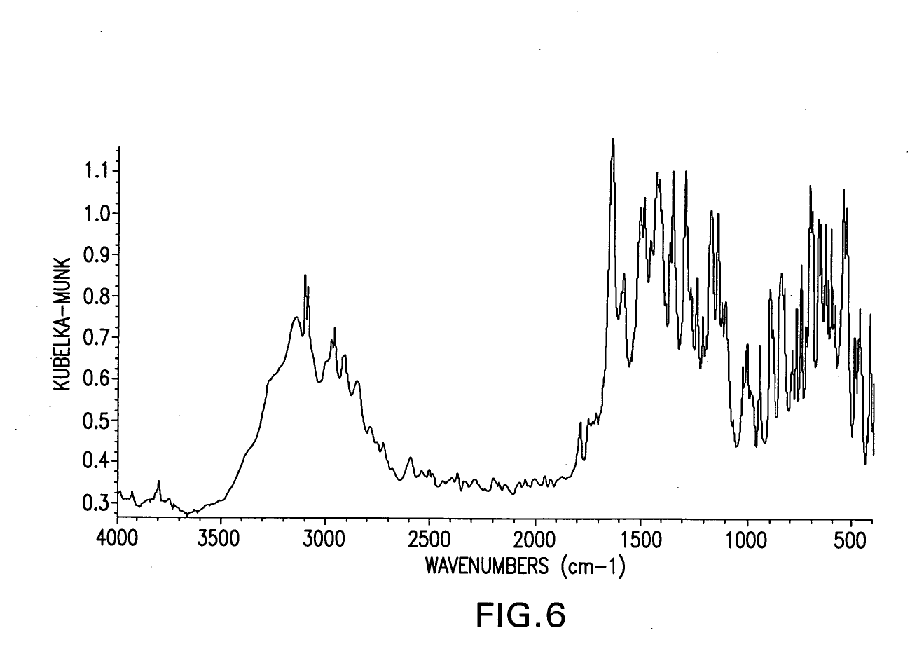

FIG. 7 is the Raman absorption spectra of the polymorph A.

FIG. 8 is the DSC of the polymorph A.

FIG. 9 is the TG of the polymorph A.

FIG. 10 is the moisture sorption/desorption of the polymorph A.

FIG. 11 is the XRPD pattern of the polymorph C.

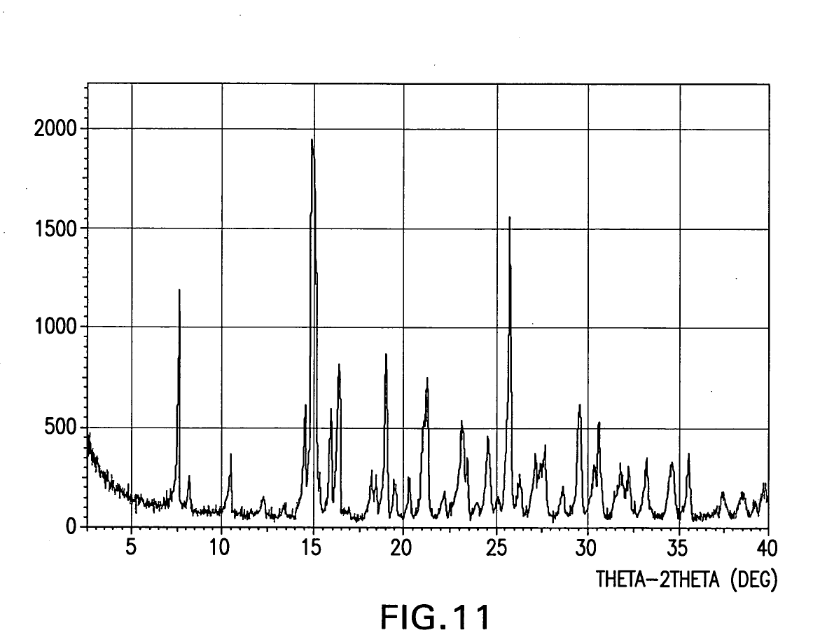

FIG. 12 is the TG/IR absorption spectra of the polymorph C.

FIG. 13 is the Raman absorption spectra of the polymorph C.

FIG. 14 is the DSC of the polymorph C.

FIG. 15 is the TG of the polymorph C.

FIG. 16 is the moisture sorption/desorption of the polymorph C.

FIG. 17 is the XRPD pattern of the polymorph E.

FIG. 18 is the TG/IR absorption spectra of the polymorph E.

FIG. 19 is the Raman absorption spectra of the polymorph E.

FIG. 20 is the DGC of the polymorph E.

FIG. 21 is the TG of the polymorph E.

FIG. 22 is the moisture sorption/desorption of the polymorph E.

FIG. 23 is TG/DSC of form D.

FIG. 24 is the moisture sorption/desorption of form D.

FIG. 25 is the DSC of the amorphous form.

FIG. 26 is the TG of the amorphous form.

FIG. 27 is the moisture sorption/desorption of the amorphous form.

### DETAILED DESCRIPTION

A. Definitions

Unless defined otherwise, all technical and scientific terms used herein have the same meaning as is commonly understood by one of skill in the art to which this invention belongs. All patents and publications referred to herein are incorporated by reference.

As used herein, an endothelin-mediated condition is a condition that is caused by abnormal endothelin activity or one in which compounds that inhibit endothelin activity have therapeutic, use. Such diseases include, but are not limited to hypertension, cardiovascular disease, asthma, inflammatory diseases, ophthalmologic disease, menstrual disorders, obstetric conditions, gastroenteric disease, renal failure, pulmonary hypertension, endotoxin shock, anaphylactic shock, or hemorrhagic shock. Endothelin-mediated conditions also include conditions that result from therapy with agents, such as erythropoietin and immunosuppressants, that elevate endothelin levels.

As used herein an effective amount of a compound for treating a particular disease is an amount that is sufficient to ameliorate, or in some manner reduce the symptoms associated with the disease. Such amount may be administered as a single dosage or may be administered according to a regimen, whereby it is effective. The amount may cure the disease or is administered in order to ameliorate the symptoms of the disease. In certain embodiments, repeated administration is required to achieve the desired amelioration of symptoms.

As used herein, an endothelin agonist is a compound that potentiates or exhibits a biological activity associated with or possessed by an endothelin peptide.

As used herein, an endothelin antagonist is a compound that inhibits endothelin-stimulated vasoconstriction and contraction and other endothelin-mediated physiological responses. The antagonist may act by interfering with the interaction of the endothelin with an endothelin-specific receptor or by interfering with the physiological response to or bioactivity of an endothelin isopeptide, such as vasoconstriction. Thus, as used herein, an endothelin antagonist interferes with endothelin-stimulated vasoconstriction or other response or interferes with the interaction of an endothelin with an endothelin-specific receptor, such as ETA receptors, as assessed by assays known to those of skill in the art.

The effectiveness of potential agonists and antagonists can be assessed using methods known to those of skill in the art. For example, endothelin agonist activity can be identified by its ability to stimulate vasoconstriction of isolated rat thoracic aorta or portal vein ring segments (Borges et al. (1989) “Tissue selectivity of endothelin” Eur. J. Pharmacol. 165: 223-230). Endothelin antagonist activity can be assessed by the ability to interfere with endothelin-induced vasoconstriction. As noted above, the preferred IC₅₀ concentration ranges are set forth with reference to assays in which the test compound is incubated with the ET receptor-bearing cells at 4° C. Data presented for assays in which the incubation step is performed at the less preferred 24° C. are identified. It is understood that for purposes of comparison, these concentrations are somewhat higher than the concentrations determined at 4° C.

As used herein a sulfonamide that is ETA selective refers to sulfonamides that exhibit an IC₅₀ that is at least about 10-fold lower with respect to ETA receptors than ETB receptors.

As used herein, a sulfonamide that is ETB, selective refers to sulfonamides that exhibit an IC₅₀ that is at least about 10-fold lower with respect to ETB, receptors than ETA receptors.

As used herein, pharmaceutically acceptable salts, esters, hydrates, solvates or other derivatives of the compounds include any such salts, esters and other derivatives that may be prepared by those of skill in this art using known methods for such derivatization and that produce compounds that may be administered to animals or humans without substantial toxic effects and that either are pharmaceutically active or are prodrugs. Pharmaceutically-acceptable salts include, but are not limited to, salts of alkali metals and alkaline earth metals, including but not limited to sodium salts, potassium salts, lithium salts, calcium salts and magnesium salts; transition metal salts, such as zinc salts, copper salts and aluminum salts; polycationic counter ion salts, such as but not limited ammonium and substituted ammonium salts and organic amine salts, such as hydroxyalkylamines and alkylamines; salts of mineral acids, such as but not limited to hydrochlorides and sulfates, salts of organic acids, such as but not limited acetates, lactates, malates, tartrates, citrates, ascorbate, succinates butyrate, valerate and fumarates. Also contemplated herein are the corresponding esters.

As used herein, reference to “sodium salts” refers to salts of any sodium compounds in which the counter ion includes Na⁺ and can include other ions, such as HPO₄²; reference to a “sodium salt” (rather than sodium salts) refers specifically to a salt in which Na⁺ is the counter ion.

As used herein, treatment means any manner in which the symptoms of a conditions, disorder or disease are ameliorated or otherwise beneficially altered. Treatment also encompasses any pharmaceutical use of the compositions herein, such as use as contraceptive agents.

As used herein, amelioration of the symptoms of a particular disorder by administration of a particular pharmaceutical composition refers to any lessening, whether permanent or temporary, lasting or transient that can be attributed to or associated with administration of the composition.

As used herein, increased stability of a formulation means that the percent of active component present in the formulation, as determined by assays known to those of skill in the art, such as high performance liquid chromatography, gas chromatography and the like, at a given period of time following preparation of the formulation is significantly higher than the percent of active component present in another formulation at the same period of time following preparation of the formulation. In this case, the former formulation is said to possess increased stability relative to the latter formulation.

B. Methods of Analysis

Crystallized samples of N-(2-acetyl-4,6-dimethylphenyl)-3-{[(3,4dimethyl-5-isoxazolyl)-amino]sulfonyl}-2-thiophenecarboxamide, were analyzed by their XRPD, infrared absorption specta, Raman spectra, melting points, differential scanning calorimetry (DSC), thermogravimetry (TG), hot-stage microscopy and automated moisture sorption/desorption to determine their polymorphic forms (Forms A, C or E) and hydrates.

1. XRPD

The XRPD analysis was carried out on a Shimadzu XRD-6000 X-ray powder diffractometer using Cu Ka radiation. The instrument was equipped with a fine-focus X-ray tube. The tube power and amperage were set at 40 kV and 40 mA, respectively. The divergence and scattering slits were set at 1° and the receiving slit was set at 0.15 mm. Diffracted radiation was detected by a Nal scintillation detector. A theta-two theta continuous scan at 3°/min (0.4 sec/0.02° step) from 2.5 °2 theta to 40 °2 theta was used. A silicon standard was analyzed each day to check the instrument alignment. Each sample was prepared for analysis by placing it in a quartz sample holder. Samples were analyzed with spinning (25 rpm) in order to reduce the effects of preferred orientation. The scan run was adjusted to 0.5°/min to correct for the spin rate.

2. Infrared Absorption

The infrared absorption spectra were acquired on a Nicolet model 860 Fourier transform infrared (FT-IR) spectrophotometer. This instrument was equipped with a globar source, a Ge/KBr beamsplitter, a deuterated triglycerine sulfate (DTGS) detector and a Spectra-Tech, Inc. diffuse reflectance accessory, which was utilized for sampling. Each spectrum represents 512 co-added scans at a spectral resolution of 4 cm⁻¹. Sample preparation consisted of mixing approximately 3 to 6 mg of a sample with potassium bromide and placing the mixture into a sample cup. A background data set was acquired with potassium bromide. A single beam sample data set was then acquired and the data plotted using kubelka-Munk units. The spectrophotometer was calibrated (wavelength) with polystyrene at the time of use.

3. Raman Spectra

The Raman spectra were acquired on a Raman bench interfaced to a Nicolet Magna 860 FT-IR spectrophotometer. This instrument utilized an excitation wavelength of 1064 nm and approximately 0.5 W of Nd:YAG laser power. The spectra represent 32 or 64 co-added scans acquired at 4 cm⁻¹ resolution. The samples were prepared for analysis by placing 3 to 6 mg of a sample in a glass tube and positioning the tube in the spectrophotometer. The spectrophotometer was calibrated (wavelength) with sulfur and cyclohexane at the time of use.

4. Differential Scanning Calorimetry (DSC)

The differential scanning calorimetry data was obtained on a TA Instruments Differential Scanning Calorimeter 2920. The calibration standard used was indium. Approximately 3 to 6 mg of a sample was placed into a DSC pan and the weight was accurately measured and recorded. The pan was sealed and a pinhole was used to allow for pressure release. The sample was heated under nitrogen at a rate of 10° C./min, up to a final temperature of 190, 200, 250, 300 or 350° C. For studies of the glass transition temperature (T_{g}) of the amorphous material, the sample was heated under nitrogen at a rate of 10°/min, up to 125° C. The sample was held at this temperature for 15 minutes and then allowed to cool and equilibrate at 25° C. The sample was again heated at a rate of 10° C./min, up to 125° C., held at this temperature for 15 minutes and then cooled and equilibrated at 25° C. for 15 minutes. The sample was then heated at 10° C./min, up to a final temperature of 250° C. The experiment was repeated using an initial temperature of −5° C., cycling up to 100° C. and reaching a final temperature of 175° C.

5. Thermogravimetric (TG) Analysis

The thermogravimetric (TG) analysis of the samples was carried out on a TA Instruments Thermogravimetric Analyzer 2050 or 2950. The calibration standards used were nickel and Alumel™. Approximately 4 to 11 mg of a sample was placed in the pan, accurately weighed and inserted into the TG furnace. The sample was then heated in nitrogen at a rate of 10° C./min, up to a final temperature of 350° C.

7. Hot-Stage Microscopy

The hot-stage microscopy was carried out on a Kofler hot-stage mounted on a Leica Microscope. The temperature of the hot-stage was measured using a Testo 6000-903 thermocouple and a Testo 720 digital readout. Temperatures were calibrated using USP standards.

8. Moisture Sorption/Desorption

The moisture sorption/desorption data was collected on a VT SGA-100 moisture balance system. For sorption isotherms, a sorption range of 5 to 95% relative humidity (RH) and a desorption range of 95 to 5% RH in 10% RH increments were used for analysis. The sample was not dried prior to analysis. Equilibrium criteria used for analysis was less than 0.0100% weight change in 5 minutes with a maximum equilibration time of 3 hours if the weight criterion was not met. Data was not corrected for the initial moisture content of the samples.

9. Polymorph Screen

A polymorph screen was undertaken in an attempt to generate as many solid forms of N-(2-acetyl-4,6-dimethylphenyl)-3-{[(3,4dimethyl-5-isoxazolyl)-amino]sulfonyl}-2-thiophenecarboxamide, as possible. This technique involved the generation of solids under a variety of conditions and subsequent characterization by XRPD. Many of the solidified samples formed exhibited preferred orientation, which is the tendency for crystals, usually plates or needles, to align themselves with some degree of order. Preferred orientations can affect peak intensities, but not peak positions in XRPD patterns.

10. Stress Studies

Samples of N-(2-acetyl-4,6-dimethylphenyl)-3-{[(3,4-dimethyl-5-isoxazolyl)amino]-sulfonyl}-2-thiophenecaboxamide were stressed at 22, 58, 75, and 93% RH under ambient temperature for 3 days. One sample was also placed in a 40 ° C. oven for 11 days. Samples of amorphous material were also placed in a 40 ° C. oven for 36 days or in a 70 ° C. oven for 4 days. The solids were then analyzed by XRPD.

11. Grinding Experiments

N-(2-acetyl-4,6-dimethylphenyl)-3-{[(3 ,4-dimethyl-5-isoxazolyl)amino]-sulfonyl}-2-thiophenecaboxamide material was ground by hand with a mortar and pestle for 30 sec, 1, 2, and 5 minutes. The samples were then analyzed by XRPD.

12. Interconversion Experiments

Interconversion experiments were carried out by making slurries containing two forms of N-(2-acetyl-4,6-dimethylphenyl)-3 -{[(3,4-dimethyl-5-isoxazolyl)amino]-sulfonyl}-2-thiophenecaboxamide in saturated toluene, methanol/water, or alcohol solutions. The slurries were agitated for various time periods at either ambient temperature or at 45° C. The insoluble solids were recovered by filtration and analyzed using XRPD.

13. Crystallization Procedures

Weighed samples of N-(2-acetyl-4,6-dimethylphenyl)-3-{[(3,4dimethyl-5-isoxazolyl)-amino]sulfonyl}-2-thiophenecarboxamide (20 to 30 mg) were treated with aliquots of a test solvent (reagent grade or HPLC grade) to provide 100 to 150 μL solutions. These solutions were sonicated and when all the solids dissolved (visual inspection), the solutions were filtered and left in an open vial under ambient conditions (fast evaporation) or were covered with aluminum foil containing pin holes (slow evaporation). Solids were removed by filtration, air-dried and analyzed by XRPD. Solid samples were also generated by rapidly cooling the above filtered, room temperature solutions to −78° C. (crash cool). Solids were removed by filtration, air-dried and analyzed by XRPD.

Weighed samples of N-(2-acetyl-4,6-dimethylphenyl)-3-{[(3,4dimethyl-5-isoxazolyl)-amino]sulfonyl}-2-thiophenecarboxamide, were also treated with aliquots of a test solvent at elevated temperatures. These samples and solvents were heated on a hotplate held at either 45° C. or 60° C. and the resulting solution was rapidly filtered into a vial kept on the same hotplate. The heat source was turned off and the hotplate and vial were allowed to cool to ambient temperature (slow cool) and allowed to stand overnight. The presence or absence of undissolved solids was noted; if there were no solids present, or an amount of solid judged too small for XRPD analysis, the vial was placed in a refrigerator overnight. Again the presence or absence of undissolved solids was noted and if there were none, the vial was placed in a freezer overnight. Solids were removed by filtration, air-dried and analyzed by XRPD.

Slurry experiments were carried out by making saturated solutions of N-(2-acetyl-4,6-dimethylphenyl)-3-{[(3,4dimethyl-5-isoxazolyl)-amino]sulfonyl}-2-thiophenecarboxamide, which contained excess solids. These slurries were agitated at ambient temperature for 10 to 20 days. The insoluble solids were removed by filtration, air-dried and analyzed by XRPD.

Antisolvent experiments were carried out by dissolving solid samples of N-(2-acetyl-4,6-dimethylphenyl)-3-{[(3,4dimethyl-5-isoxazolyl)-amino]sulfonyl}-2-thiophenecarboxamide, in a test solvent and filtering the resulting solution into an antisolvent cooled to approximately 0° C. If no solids immediately formed, the samples were left under ambient conditions until solids were seen. Solids were removed by filtration, air-fried and analyzed by XRPD.

Vapor diffusion experiments were carried out by placing a saturated solution of N-(2-acetyl-4,6-dimethylphenyl)-3-{[(3,4dimethyl-5-isoxazolyl)-amino]sulfonyl}-2-thiophenecarboxamide in a vial which was placed in a larger vial containing an antisolvent. The larger vial was sealed and kept at ambient temperature. Solids were removed by filtration, air-dried and analyzed by XRPD.

Liquid diffusion experiments were carried out by placing a saturated solution of N-(2-acetyl-4,6-dimethylphenyl)-3-{[(3,4dimethyl-5-isoxazolyl)-amino]sulfonyl}-2-thiophenecarboxamide, in a vial and adding an immiscible antisolvent. The presence or absence of precipitated solids was noted. If solids formed, the solvents were decanted and the solids collected. If no solids formed, the vial was capped and left to stand at ambient temperature. Any solids formed were removed by filtration, air-dried and analyzed by XRPD.

A solid sample was also generated by quickly cooling (−78° C.) a melt of N-(2-acetyl-4,6-dimethylphenyl)-3-{[(3,4dimethyl-5-isoxazolyl)-amino]sulfonyl}-2-thiophenecarboxamide and was analyzed by XRPD.

C. Polymorphs A, C, E and an Amorphous Material

The solid forms obtained in the polymorph screen of N-(2-acetyl-4,6-dimethylphenyl)-3-{[(3,4dimethyl-5-isoxazolyl)-amino]sulfonyl}-2-thiophenecarboxamide, are summarized in Table 1. Three distinct XRPD patterns representing three distinct forms, designated as Forms A, C and E were found. Form A was obtained by slow evaporations, slow cools and vapor diffusion crystallizations. Form C was obtained from slow evaporations from toluene solutions. Form E was obtained from fast evaporations of solutions or antisolvent crystallizations. The amorphous material was obtained by rapidly cooling (−78° C.) a melt of this compound.

TABLE 1

| | | CRYSTAL | |
|---|---|---|---|
| SOLVENT | METHOD | HABIT | XRPD |
| acetone | FE | clear solid with | A + E |
| | | white solid | |
| | | inside it | |
| acetone | SE | clear fibrous | A(PO) |
| | | crystals | |
| acetone | SC(60° C.) | no solid | — |
| acetone | CC | no solid | — |
| acetonitrile | FE | clear fibrous & | A(PO) |
| | | prismatic crystals | |
| acetonitrile | SE | clear prismatic & | A |
| | | acicular crystals | |
| acetonitrile | SC(60° C.) | no solids | — |
| dichloromethane | FE | white solid | A(PO) |
| dichloromethane | SE | clear acicular | A, SS |
| | | crystals | |
| | | & white solid | |
| diethyl ether | SE | yellow solid | A(PO) |
| diethyl ether | slurry | light yellow solid | A(PO) |
| N.N- | FE | yellow solid | A(PO) |
| dimethylformamide | | | |
| N.N- | SE | clear yellow | C + amorphous + |
| dimethylformamide | | prisms | E |
| dimethyl sulfoxide | FE | yellow oil | — |
| dimethyl sulfoxide | SE | yellow oil | — |
| ethanol | FE | clear tablet, | E + A |
| | | prismatic, plate | |
| | | and acicular | |
| | | crystals | |
| ethanol | SE | clear | A(PO) |
| | | prismatic/tabular | |
| | | crystals | |
| ethyl acetate | FE | white fibrous | E(PO) |
| | | crystals | |
| ethyl acetate | FE | — | C + E |
| ethyl acetate | FE | — | E + A(min) |
| ethyl acetate | SE | clear plate and | A(PO) + E(PO) |
| | | acicular crystals | |
| ethyl acetate | SC(60° C.) | yellow solid | A(PO) |
| ethyl acetate | CC | no solid | — |
| hexanes | SE | yellow solid | A |
| hexanes | slurry | light yellow solid | A |
| isopropanol | SE | yellow solid | A |
| isopropanol | slurry | light yellow solid | A |
| methanol | FE | clear acicular | E |
| | | crystals, clear | |
| | | solid | |
| methanol | FE | — | A + E(min) |
| methanol | SE | yellow solid | A(PO) |
| methanol | SC(60° C.) | yellow solid | A(PO), SS |
| methanol | CC | no solid | — |
| methanol | rotovap | — | amorphous SS |
| methanol/water | slurry | yellow solid | A + C |
| (1:1) | | | |
| 1-propanol | SE | white solid | A(PO) |
| 1-propanol | slurry | light yellow solid | A |
| tetrahydrofuran | FE | white solid | E |
| tetrahydrofuran | SE° | white solid | amorphous |
| tetrahydrofuran | CC | no solid | — |
| toluene | FE | clear fibrous | E + A(PO) |
| | | crystals, unknown | |
| | | white solid | |
| toluene | SE | yellow solid | C(PO) + E(min) |
| toluene | SE | — | C |
| toluene | SE | — | A |
| toluene | SE | — | A(PO) |
| toluene | SE | — | PO |
| toluene | SE | — | A(PO) |
| toluene | SC(60° C.) | yellow solid | A + C |
| toluene | slurry | — | A |
| toluene | CC | no solid | — |
| water | SE | white solid | A |
| water | slurry | yellow solid | A |
| — | CC of melt | yellow solid | amorphous |

^{a}FE = fast evaporation;
SE = slow evaporation;
SC = slow cool;
rotovap = rotary evaporation;
grd = ground
CC = crash cool to −78° C.
^{b}PO = preferred orientation;
SS = small sample;
IS = insufficient sample;
^{c}samples placed in oven to dry

### Crystallization Studies

Crystallization studies and detailed processes for preparing polymorphs of N-(2-acetyl-4,6-dimethylphenyl)-3-{[(3,4dimethyl-5-isoxazolyl)-amino]sulfonyl}-2-thiophenecarboxamide, in Forms A, C and E are described below. These studies demonstrate that polymorphs A, C and E can be selectively produced under appropriate conditions. Further, these forms, and mixtures thereof, can be interconverted to Form A. In contrast the metastable Form E appears to be kinetically favored.

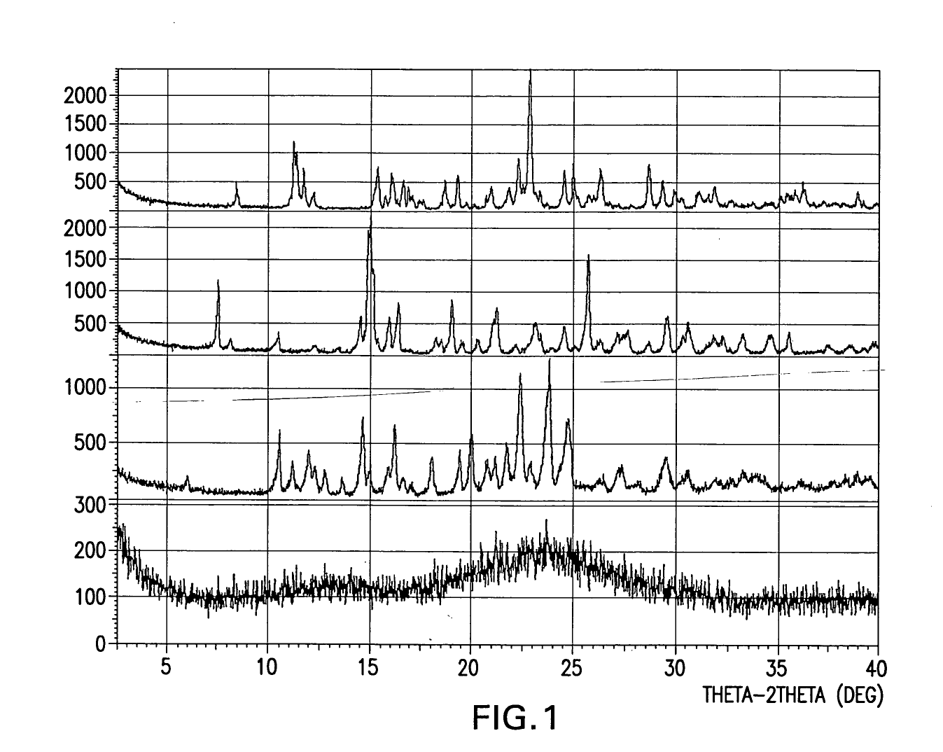

The XRPD patterns of the solid crystalline form of N-(2-acetyl-4,6-dimethylphenyl)-3-{[(3,4dimethyl-5-isoxazolyl)-amino]sulfonyl}-2-thiophenecarboxamide, in Forms A, C and E are shown in FIGS. 1, 4 and 7 respectively. These XRPD patterns were used to identify the solid forms obtained from the crystallization and process studies described below.

a. 75° C. to 53° C. to Room Temperature

A saturated solution of N-(2-acetyl-4,6-dimethylphenyl)-3-{[{3,4dimethyl-5-isoxazolyl)-amino]sulfonyl}-2-thiophenecarboxamide in ethanol at 75° C. was slowly cooled and allowed to evaporate in an uncapped vial. At 53° C., solids first appeared in the solution. After 1 hour, the vial was capped and was stored at room temperature for 6 hours. Periodically, samples of the solid were removed, recovered by vacuum filtration and analyzed by XRPD. The initial sample and all subsequent samples of the solids were found to be of Form A. These results demonstrate that these conditions favor the spontaneous crystallization of Form A.

b. 75° C., Reduce Volume, Cool to 5° C.

A saturated solution of N-(2-acetyl-4,6-dimethylphenyl)-3-{((3,4dimethyl-5-isoxazolyl)-amino]sulfonyl}-2-thiophenecarboxamide in ethanol at 75° C. was kept at that temperature in an uncapped vial until the total volume was reduced by 25%. The vial was then capped and placed in a 5° C. water bath for 6 hours. Solids first appeared in the solution at 5° C. Periodically, samples of the solids were removed and were recovered by vacuum filtration and analyzed by XRPD. The initial sample and all subsequent samples of the solids were found to be of Form E. The interconversion of these solids to Form A was not observed under these conditions. These results demonstrate that these conditions favor the spontaneous crystallization of the metastable Form E.

c. 75° C. to 45° C., Hold Until Solids Form, Cool to 5° C.

A saturated solution of N-(2-acetyl-4,6-dimethylphenyl)-3-([(3,4dimethyl-5-isoxazolyl)-aminol]sulfonyl}-2-thiophenecarboxamide in ethanol at 75° C. was slowly cooled to 45° C. and was allowed to evaporate in an uncapped vial. At 45° C., solids first appeared in the solution. The vial was capped and placed in a 5° C. water bath for 6 hours. The solids, which were removed and were recovered by vacuum filtration, were all of Form A. Cooling the vial to 5° C. after solids appear does not result in a formation of Form E under these conditions. These results demonstrate that once Form A is crystallized, the solids will remain in Form A over a wide temperature range.

d. 75° C. to 45° C., Seed with Forms A and E

A saturated solution of N-(2-acetyl-4,6-dimethylphenyl)-3-([(3,4dimethyl-5-isoxazolyl)-amino]sulfonyl}-2-thiophenecarboxamide in ethanol at 75° C. was slowly cooled to 45° C. and was allowed to evaporate in an uncapped vial. Seed crystals of Forms A and E were added to the clear solution and the vial was held at 45° C. for 6 hours. Periodically, samples of the solids were removed and were recovered by vacuum filtration and analyzed by XRPD. After 1 minute, the solids consisted of Forms A and E. After 5 minutes, however, the solids were only of Form A. These results demonstrate that even when Form E is initially present, Form E solids are interconverted to Form A.

e. 75° C. to 45° C., Seed with Form E

A solution of N-(2-acetyl-4,6-dimethylphenyl)-3-{((3,4dimethyl-5-isoxazolyl)-amino)sulfonyl)-2-thiophenecarboxamide in ethanol at 75° C. was slowly cooled to 45 ° C. and was allowed to evaporate in an uncappped vial. Seed crystals of Form E were added to the clear solution and the vial was held at 45° C. for 6 hours. Solids first appeared in the solution approximately 15 minutes after these seeds were added. Periodically, samples of the solids were removed and were recovered by vacuum filtration and analyzed by XRPD. In all cases, only Form A was recovered. These results demonstrate that even when only Form E seed crystals are present at 45° C., only Form A is formed.

f. 75° C. to 45° C., Seed with Form E, Reduce Volume

A saturated solution of N-(2-acetyl-4,6-dimethylphenyl)-3-{[(3,4dimethyl-5-isoxazolyl)-amino]sulfonyl}-2-thiophenecarboxamide in ethanol at 75° C. was slowly cooled and was allowed to evaporate in an uncapped vial. At 45° C., seed crystals of Form E were added to the clear solution. The vial was kept at 45° C. and the volume of the solution was reduced by 25% over 1 hour. Periodically, samples of the solids were removed and were recovered by vacuum filtration and analyzed by XRPD. The initial samples and all subsequent samples of the solids were found to be of Form A. These results demonstrate that a supersaturated solution containing Form E seed crystals at 45° C., does not result in the metastable Form E being formed.

g. 75° C. to 45° C., Seed with Form C

A saturated solution of N-(2-acetyl-4,6-dimethylphenyl)-3-{[(3,4dimethyl-5-isoxazolyl)-amino)sulfonyl}-2-thiophenecarboxamide in ethanol at 75° C. was slowly cooled and was allowed to evaporate in an uncapped vial. At 450C, seed crystals of Form C were added to the clear solution. The vial was kept at 45° C. and the volume of the solution was reduced by 25% over 1 hour. Periodically, samples of the solids were removed and were recovered by vacuum filtration and analyzed by XRPD. After 10 minutes, the solids were of Forms A and C. After 25 minutes, however, the solids were found to be only of Form A. These results demonstrate that Form C will convert to Form A at elevated temperatures (45° C.).

h. 5° C., Seed with Forms A and E

A saturated solution of N-(2-acetyl-4,6-dimethyiphenyl)-3-{[(3,4dimethyl-5-isoxazolyl)-aminolsulfonyl}-2-thiophenecarboxamide in ethanol at 5° C. was filtered and seed crystals of Forms A and E were added. The vial was capped and stored at 5° C. for 6 hours. Periodically, samples of the solids were removed and were recovered by vacuum filtration and analyzed by XRPD. After 1 minute, the solids were a mixture of Form A and possibly Form E. After 6 minutes, any Form E that may have been present in the solids converted to Form A. These results demonstrate that the rate of conversion of Form E to Form A does not appear to be dependent on temperature when Form A seeds are present.

Summary of Crystallization Studies

Based on the experiments performed, crystallization of a saturated solution of N-(2-acetyl-4,6-dimethylphenyl)-3-{[(3,4dimethyl-5isoxazolyl)-amino]sulfonyl}-2-thiophenecarboxamide in ethanol favors the formation of Form A at elevated temperatures and when Form A seeds are present. If a saturated solution of this compound in ethanol is allowed to crystallize at low temperatures (5° C.) without any seed crystals being present, Form E is produced. Form E appears to persist and does not readily convert to Form A under these conditions (5° C.). Experiments conducted at all temperatures which involved Forms A or C seed crystals resulted in the crystallization of Form A. However, the formation of Form A was slower at lower temperatures.

It appears that metastable Form E is the kinetically favored form at low temperatures (5° C.). To ensure the production of Form A, saturated solutions of N-(2acetyl-4,6-dimethylphenyl)-3-{[(3,4dimethyl-5-isoxazolyi)amino]sulfonyl}-2-thiophenecarboxamide in ethanol should be stirred at 45° C. for an extended period of time and then seeded with Form A crystals at this temperature, before the appearance of any solids.

2. Solubility Studies

The solubility of N-(2-acetyl-4,6-dimethylphenyl) -3-{t(3,4dimethyl5-isoxazolyi)-amino]sulfonyl}-2-thiophenecarboxamide in Forms A, C and E in ethanol were determined and the data are summarized in Table 2. Saturated solutions of this compound in ethanol were prepared and each solution was heated to either 25, 27, 35, 40 or 50° C.' and stirred for 30 minutes. The samples were filtered into a receiving flask and were heated at the same temperatures and stirred for another 30 minutes. Samples were removed from these flasks and were filtered into a tared vial. The remaining solution was heated and stirred for another 30 minutes. Samples were again removed from these flasks and were filtered into another tared vial. The solvents in the tared vials were removed using a rotary evaporator and the weights of the solids in the tared vials were recorded and were used to calculate solubility.

TABLE 2

| | | | AVERAGE |
|---|---|---|---|
| STARTING | TEMPERATURE | | SOLUBILITY |
| FORM | ° C. | TIME (min) | (mg/mL) |
| A | 25 | 30 | 9.5 |
| A | 25 | 60 | 10.6 |
| A | 40 | 30 | 17.3 |
| A | 40 | 60 | 18.1 |
| A | 50 | 30 | 40.8 |
| A | 50 | 60 | 36.7 |
| C | 25 | 30 | 16.1 |
| C | 25 | 60 | 13.2 |
| C | 40 | 30 | 28.3 |
| C | 40 | 60 | 27.4 |
| C | 50 | 30 | 35.6 |
| C | 50 | 60 | 45.1 |
| E | 27 | 30 | 12.7 |
| E | 27 | 60 | 11.0 |
| E | 35 | 30 | 17.7 |
| E | 35 | 60 | 18.3 |
| E | 50 | 30 | 40.8 |
| E | 50 | 60 | 39.7 |

The solubilitites reported were determined gravimetrically, therefore, fluctuations in the data are possible. Minor differences were observed between 30 and 60 minute timepoints, indicating that 30 minutes was sufficient to reach equilibrium in most cases. Form A appears to be the less soluble form. XRPD data collected on the remaining solid show that conversion to another form did not occur during the solubility experiment. The solubility values for Form E are similar to Form A. Form E converted to Form A during the experiment based on the XRPD pattern, therefore, the solubilities observed for Form E represent Form A or a mixture of Forms E and A. In general, Form C material exhibited a higher solubility. XRPD data on the remaining material confirm that the material did not change form during the experiment.

In summary, the solubility studies of N-(2-acetyl-4,6dimethylphenyl)-3-{[(3,4dimethyl-5-isoxazoiyl)-aminolsulfonyl}-2thiophenecarboxamide in ethanol at various temperatures show that Form A is the least soluble. Form E converted to Form A during the solubility experiment, therefore, the solubility of Form E was not determined. Form C exhibited a higher solubility than Form A and did not convert during the solubility experiment. Based on the solubility data, the stability of the forms was determined to be Form A >Form C >Form E.

a. Approximate Solubilities

The approximate solubilities of N-(2-acetyl-4,6-dimethylphenyl)-3{t(3,4dimethyl-5-isoxazolyl)-aminolsulfonyl}-2-thiophenecarboxamide in Form A in various solvents at ambient temperature are summarized in Table 3. The approximate solubilities were estimated from experiments based on the total solvent used to give a solution. The actual solubilities may be greater than those calculated because of the use of too-large solvent aliquots or a slow rate of dissolution. If dissolution did not occur during the experiment the solubility is expressed as “less than”. If the solid dissolved before the whole aliquot of solvent was added the solubility is listed as “greater than”.

Form A of N-(2-acetyl-4,6-dimethylphenyl)-3-{1(3,4dimethyl-5isoxazolyl)-amino]sulfonyl}-2-thiophenecarboxamide was found to be most soluble in N,N-dimethylformamide (250 mg/mL), followed by dimethylsulfoxide (144 mg/mL), dichloromethane (135 mglmL), tetrahydrofuran (132 mg/mL) and acetone (87 mg/mL), see Table 3, below. Form A was found to be sparingly soluble in diethyl other, hexanes, propanol and water.

TABLE 3

| SOLVENT | SOLUBILITY (mg/mL) |
|---|---|
| acetonitrile (CAN) | 72 |
| acetone | 87 |
| dichloromethane (CH₂CI₂) | 135 |
| diethyl ether | <9 |
| N,N-dimenthylformamide (DMF) | 250 |
| Dimethyl sulfoxide (DMSO) | 144 |
| ethanol (EtOH) | 9 |
| ethyl acetate (EtOAc) | 21 |
| hexanes | <9 |
| isopropanol (IPA) | <10 |
| methanol (MeOH) | 21 |
| propanol | <9 |
| tetrahydrofuran (THF) | 19 |
| toluene | 19 |
| water | <8 |

^{a}To determine the solubility of N-(2-acetyl-4,6-dimethylphenyl)-3-{1(3,4 dimethyl-5isoxazolyl)-amino]sulfonyl}-2-thiophenecarboxamide in various solvents, a test solvent in measured portions (usually 100 uL) was added to an accurately weighed sample with shaking, stirring or sonication at ambient temperature until a clear solution resulted.
^{b}Solvents are listed in alphabetical order
^{c}Solubilities were calculated based on the total solvent used to give a solution. Actual solubilities may be greater due to the volume of the solvent portions utilized or to a slow rate of dissolution. Values are rounded to the nearest mg/mL.

Form A

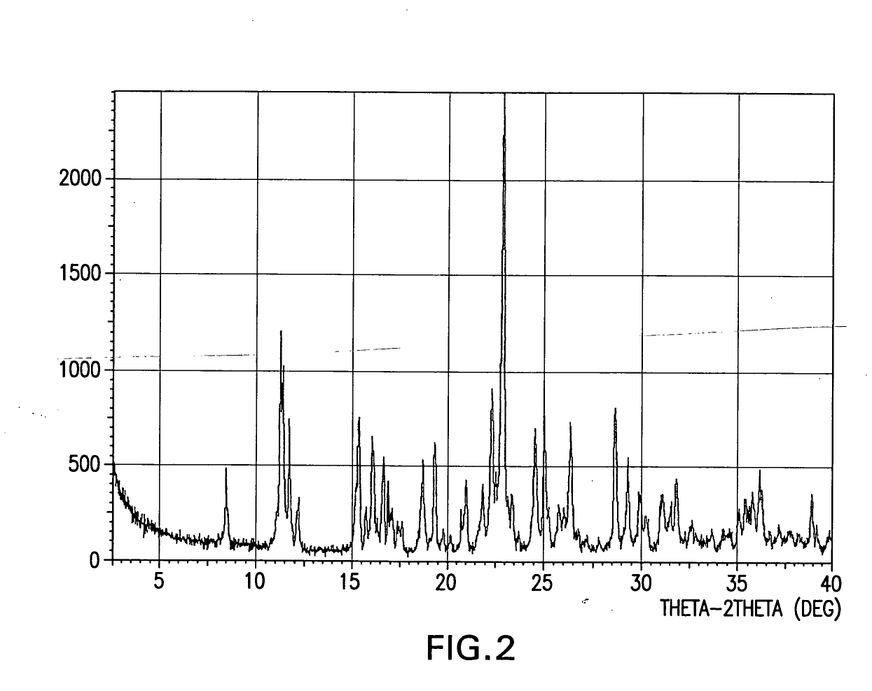

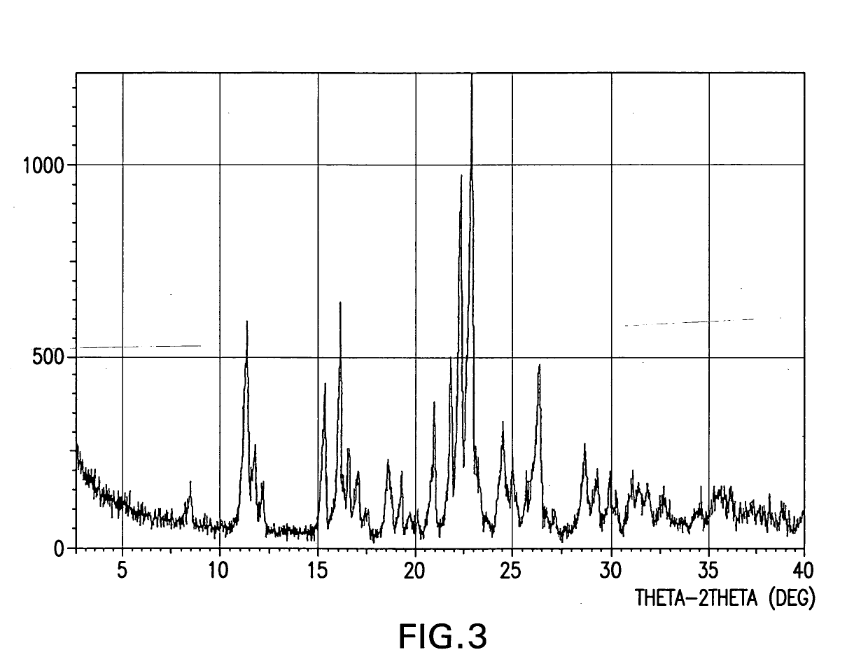

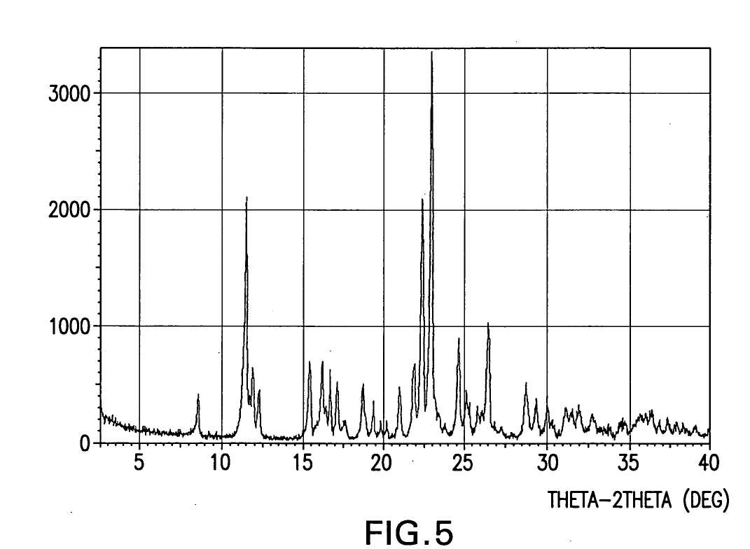

The XRPD pattern for Form A is shown in FIGS. 1-5 and Table 4 below lists peaks in the XRPD. In the polymorph screen, Form A was most often produced from slow evaporations, slow cools, and vapor diffusion crystallizations.

TABLE 4
XRPD peaks for Forms A, C and E

| Form A | Form C | Form E |
|---|---|---|
| 8.46 | 6.94 | 6.02 |
| 11.26 | 7.56 | 10.54 |
| 11.72 | 8.16 | 11.22 |
| 12.2 | 10.46 | 11.96 |
| 15.34 | 12.28 | 12.26 |
| 16.06 | 13.44 | 12.74 |
| 16.66 | 14.54 | 13.6 |
| 16.88 | 15.02 | 14.66 |
| 17.64 | 15.96 | 15.02 |
| 18.7 | 16.4 | 15.92 |
| 19.32 | 18.26 | 16.2 |
| 20.7 | 19.04 | 16.66 |
| 20.98 | 19.52 | 17.08 |
| 21.82 | 20.32 | 18.08 |
| 22.32 | 21.24 | 19.44 |
| 22.9 | 22.22 | 20.04 |
| 23.36 | 23.12 | 20.84 |
| 24.56 | 24.56 | 21.18 |
| 25.02 | 25.74 | 21.8 |
| 25.76 | 26.34 | 22.44 |
| 26.34 | 27.14 | 22.94 |
| 27.22 | 27.68 | 23.82 |
| 28.68 | 28.68 | 24.82 |
| 29.32 | 29.54 | 26.52 |
| 29.88 | 30.3 | 27.4 |
| 31.1 | 30.56 | 29.5 |
| 31.5 | 31.78 | 30.6 |
| 31.82 | 32.18 | 31.94 |
| 32.66 | 33.2 | 32.74 |
| 33.68 | 34.58 | 33.22 |
| 34.32 | 35.5 | 33.82 |
| 35.06 | 37.54 | 36.14 |
| 35.34 | 38.54 | 37.82 |
| 35.78 | 39.84 | 38.36 |
| 36.18 | | 38.96 |
| 37.24 | | 39.62 |
| 37.84 | | |
| 38.96 | | |

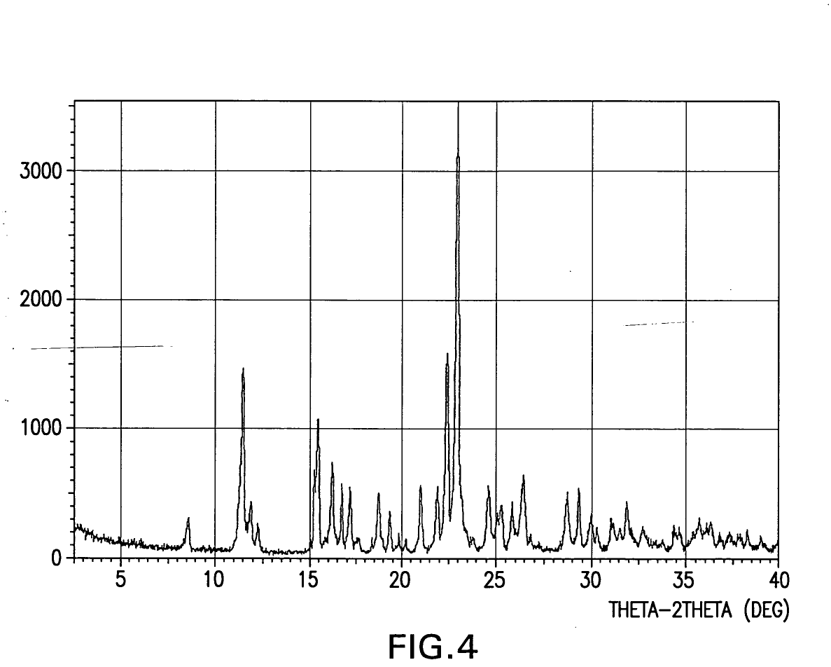

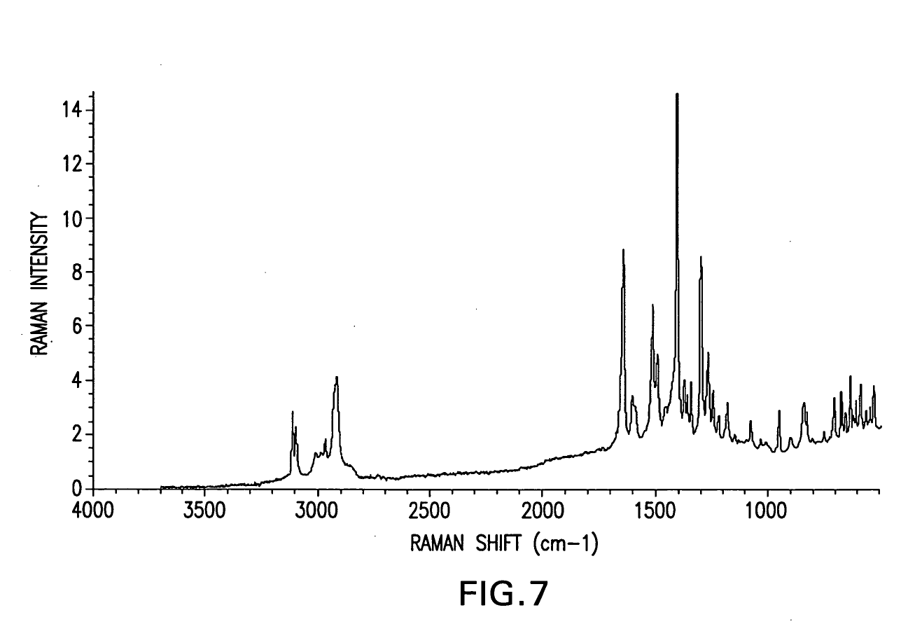

The IR and Raman data are summarized in Tables 5 and 6 and plotted in FIGS. 4 and 7, respectively. The IR spectrum of Form A has unique peaks at 3810, 3156 (broad), 1466, 1396, 1363, 1135, 999, 908, 902, and 850 cm−1 that are not found in the spectra of Forms C or E.

TABLE 5
IR peaks for Form A, C and E

| Form A | Form C | Form E |
|---|---|---|
| 3155.1 | 3919 | 3268.8 |
| 3113.9 | 3888.6 | 3129.9 |
| 3100.7 | 3782.7 | 3114.4 |
| 2986.9 | 3241.1 | 3004.8 |
| 2971 | 3116.5 | 2982.3 |
| 2920.6 | 3082.1 | 2929.3 |
| 2860.3 | 2989.7 | 2858.4 |
| 2798.7 | 2965.2 | 2762 |
| 2735.9 | 2928.6 | 2604.3 |
| 2604.3 | 2861.3 | 1769.2 |
| 1799.6 | 2738.4 | 1658.8 |
| 1764.2 | 2687.8 | 1648.8 |
| 1738 | 2612.6 | 1592.5 |
| 1726.2 | 2544.8 | 1522 |
| 1649 | 2530.4 | 1498 |
| 1593.9 | 2508.3 | 1428.8 |
| 1562.5 | 2439.4 | 1375.6 |
| 1517.7 | 2391.1 | 1356.9 |
| 1499 | 2337.4 | 1346.7 |
| 1466.8 | 2302.6 | 1304.2 |
| 1440.3 | 2271.8 | 1276.8 |
| 1427.6 | 2200.8 | 1249.2 |
| 1376 | 2167.5 | 1222.6 |
| 1362.8 | 2103.1 | 1184.3 |
| 1351 | 2068.9 | 1170.1 |
| 1304.6 | 2018.5 | 1150.8 |
| 1278.4 | 1906.5 | 1139.2 |
| 1250.9 | 1816.8 | 1119.8 |
| 1224.2 | 1771.8 | 1072.6 |
| 1187.8 | 1682.8 | 1041.9 |
| 1153.5 | 1657.4 | 1024.9 |
| 1136.4 | 1602.9 | 1012.2 |
| 1118.7 | 1589.7 | 993.5 |
| 1082.9 | 1526.1 | 959.3 |
| 1036.7 | 1494.6 | 911.7 |
| 1023.6 | 1460.8 | 891.1 |
| 1012.9 | 1425.3 | 861.6 |
| 1000.3 | 1402.3 | 837.3 |
| 957.2 | 1378.7 | 800.5 |
| 907.7 | 1358.5 | 781.7 |
| 892.1 | 1345.6 | 752.3 |
| 861.9 | 1306.2 | 732.3 |
| 850.8 | 1292.2 | 709.9 |
| 836.5 | 1272.6 | 670.4 |
| 799.3 | 1253.3 | 644.9 |
| 780.6 | 1220.5 | 622.7 |
| 755.6 | 1189.6 | 606.5 |
| 732 | 1178.5 | 590.1 |
| 711.5 | 1152.8 | 547.4 |
| 699.6 | 1140.3 | 489.5 |
| 670.8 | 1126.2 | 468.3 |
| 657.8 | 1100.7 | 439.3 |
| 636.2 | 1083.8 | 420.3 |
| 622.6 | 1036.4 | 402.4 |
| 607.8 | 1017 | |
| 588.9 | 987.4 | |
| 547.9 | 956.5 | |
| 532.5 | 917.8 | |
| 493.7 | 895.8 | |
| 469.1 | 864.3 | |
| 439.7 | 839.5 | |
| 420.1 | 811.2 | |
| 403.4 | 784.6 | |
| | 775.9 | |
| | 746 | |
| | 728.4 | |
| | 705.6 | |
| | 680.3 | |
| | 665.6 | |
| | 652.7 | |
| | 633.4 | |
| | 604.9 | |
| | 581.7 | |
| | 548.4 | |
| | 528 | |
| | 513.4 | |
| | 495 | |
| | 465.7 | |
| | 442.2 | |
| | 421.6 | |
| | 405 | |

TABLE 6
Raman peaks for Form A, C and E

| Form A | Form C | Form E |
|---|---|---|
| 3113.4 | 3116 | 3130.7 |
| 3100.7 | 3082.2 | 3114.9 |
| 3013.8 | 2988.3 | 3003.5 |
| 2971 | 2963.2 | 2924.9 |
| 2923.8 | 2928.3 | 1658.7 |
| 2768.3 | 2860.1 | 1648.2 |
| 2736.5 | 2737.3 | 1603.2 |
| 1647.4 | 1683.9 | 1523.3 |
| 1605.9 | 1654.6 | 1496 |
| 1594.1 | 1603 | 1464.1 |
| 1519.1 | 1588.8 | 1419.1 |
| 1497.8 | 1522.7 | 1386.2 |
| 1461.7 | 1494.3 | 1367.3 |
| 1414 | 1462.9 | 1357.1 |
| 1379.6 | 1417.8 | 1345.8 |
| 1367.1 | 1384.5 | 1304.8 |
| 1350 | 1358.8 | 1275 |
| 1307.4 | 1346 | 1248.5 |
| 1274.2 | 1306.7 | 1223.8 |
| 1249.3 | 1291.7 | 1182.4 |
| 1225.9 | 1272.2 | 1149.3 |
| 1187.8 | 1251.6 | 1094.4 |
| 1153.4 | 1220 | 1067 |
| 1081.9 | 1187.9 | 958.7 |
| 1037.1 | 1177.6 | 910 |
| 1013.1 | 1150.9 | 855 |
| 957.2 | 1100.1 | 837.1 |
| 907.5 | 1083.8 | 754.6 |
| 850 | 1015.9 | 709.1 |
| 836.5 | 987.6 | 664.2 |
| 810.7 | 957.9 | 644.7 |
| 755.9 | 916.8 | 607.5 |
| 710.3 | 896.5 | 592.8 |
| 678.3 | 865.8 | 549.4 |
| 659.6 | 838.9 | 533.6 |
| 638 | 783.3 | 484.4 |
| 622.7 | 745.8 | 468.2 |
| 612.2 | 727.3 | 440.2 |
| 590.8 | 705.4 | 419.7 |
| 565.4 | 680.6 | 400.1 |
| 548.6 | 663.5 | 372.4 |
| 532.1 | 625.2 | 325.3 |
| 479.9 | 581.7 | 302.7 |
| 469.3 | 545.4 | 276.3 |
| 441.7 | 514.9 | 235.5 |
| 421.2 | 492.5 | 208.6 |
| 403.7 | 464.9 | 199.5 |
| 372.1 | 442 | 169.9 |
| 323.5 | 421.3 | 160.2 |
| 297.4 | 405.9 | 123.6 |
| 274.5 | 384.9 | |
| 248.7 | 372.9 | |
| 219.6 | 355.1 | |
| 208.9 | 326.1 | |
| 171.1 | 288.5 | |
| 147.4 | 245.2 | |
| 118.9 | 226 | |
| | 193.3 | |
| | 168.4 | |
| | 136.2 | |

The Form A Raman spectrum has unique peaks at 3100, 2970, 1414, 1350, 850, and 640 cm−1 that are not found in the spectra of Forms C or E.

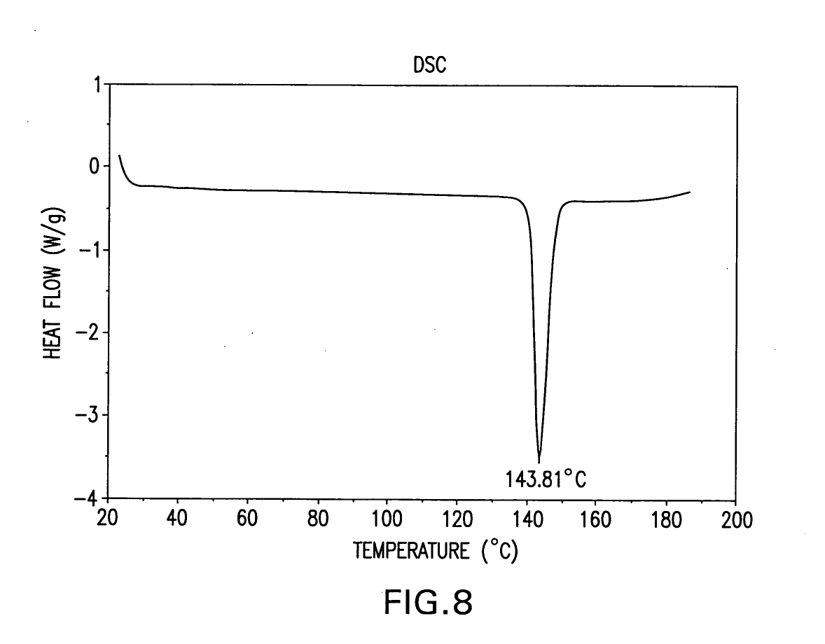

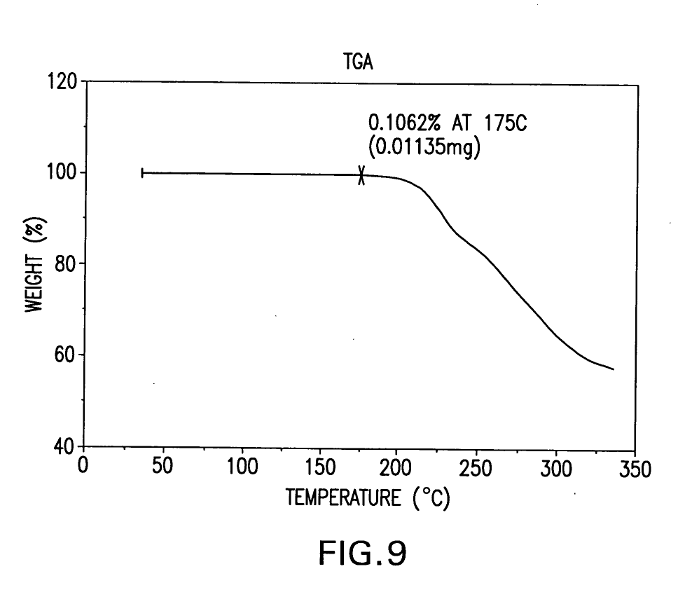

Thermal data for Form A are summarized in Tables 7 and 8 and plotted in FIGS. 8 and 9. The TG curve shows a total volatile content of 0.1% at 175° C., indicating an unsolvated material. The DSC curve exhibited an endotherm at 143.8° C., which is attributed to melting based on hot stage microscopy data (Table 8).

TABLE 7
Thermal Data for N-(2-acetyl-4,6-dimethylphenyl)-3-{[(3,4-dimethyl-5-
isoxazolyl)amino]-sulfonyl}-2-thiophenecaboxamide Forms

| | DSC Results | TG |
|---|---|---|
| Form | (° C.)^{a} | Results^{b} |
| A | endo 143.8 | 0.1 |
| C | endo 142.8 | <0.1 |
| | endo 148.7 | — |
| E | endo 148.5 | <0.1 |
| | endo 145.9 | — |
| A + E | endo 144.4, | — |
| | 149.1 | |
| Pattern D | endo 145, 150 | 0.4 |
| amorphous | endo 60.0, | — |
| | 149.3 | |
| | — | 4.0 |
| | endo 63.8, | — |
| | 149.4 | |
| | endo 57.3, | — |
| | 149.0 | |

^{a}maximum temperature reported; endo = endotherm
^{b}percent volatiles measured at 175° C.

TABLE 8
Hot Stage Studies

| Form | Observations |
|---|---|
| A | melt range 142-148° C. |
| C | melt range 143-146° C. |
| E | melt range 148-151° C. |
| Pattern D | regions melt at 140-145° C., other regions at |
| | 148-151° C. |
| amorphous | liquifies 77-80° C.; solid forms 87° C., melts |
| | at 145° C. |

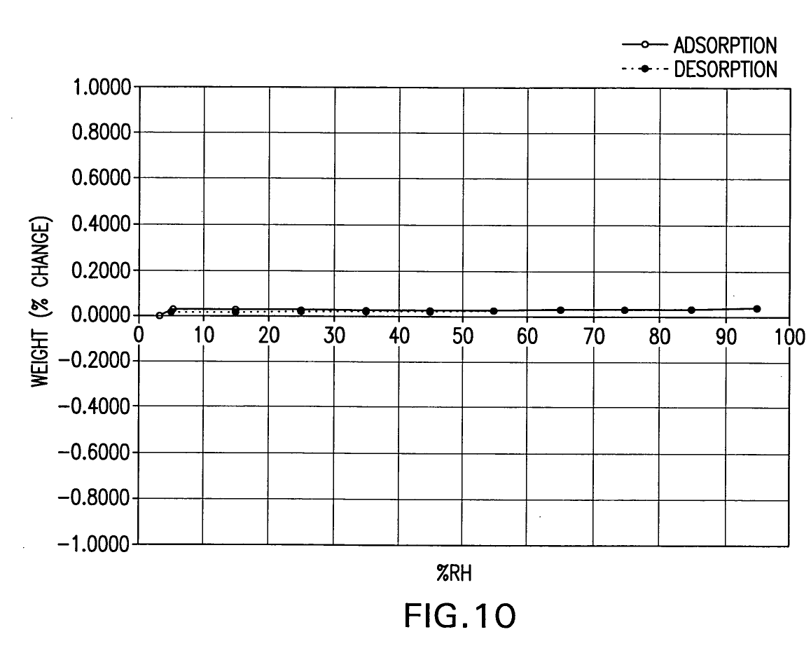

Moisture sorption/desorption data for Form A are summarized in Table 9 and plotted in FIG. 10. The material shows minimal weight loss or gain during the experiment. The XRPD pattern of the sample after the experiment was completed indicates that the material remained Form A. Based on the moisture sorption/desorption data, Form A appears to be a non-hygroscopic material.

TABLE 9
Summary of Moisture Sorption/Desorption Data for N-(2-acetyl-4,6-
dimethylphenyl)-3-{[3,4-dimethyl-5-isoxazolyl)amino]-
sulfonyl}-2-thiophene-caboxamide Forms

| | | XRPD |
|---|---|---|
| Form | Moisture Balance Results | Results |
| A | <0.1% weight loss at 5% RH, | A |
| | <0.1% total weight gain at 95% RH | |
| C | <0.1% weight loss at 5% RH, | C |
| | <0.1% total weight gain at 95% RH | |
| E | <0.1% weight loss at 5% RH, | E |
| | 0.15% total weight gain at 95% RH | |
| Pattern D | 0.47% weight loss at 5% RH, | — |
| | 1.82% total weight gain at 95% RH | |
| amorphous | <0.1% weight loss at 5% RH, | amorphous |
| | 0.99% total weight gain at 95% RH | |

Based on the characterization data, Form A appears to be an unsolvated, non-hygroscopic, crystalline material that melts at approximately 144° C.

2. Form C

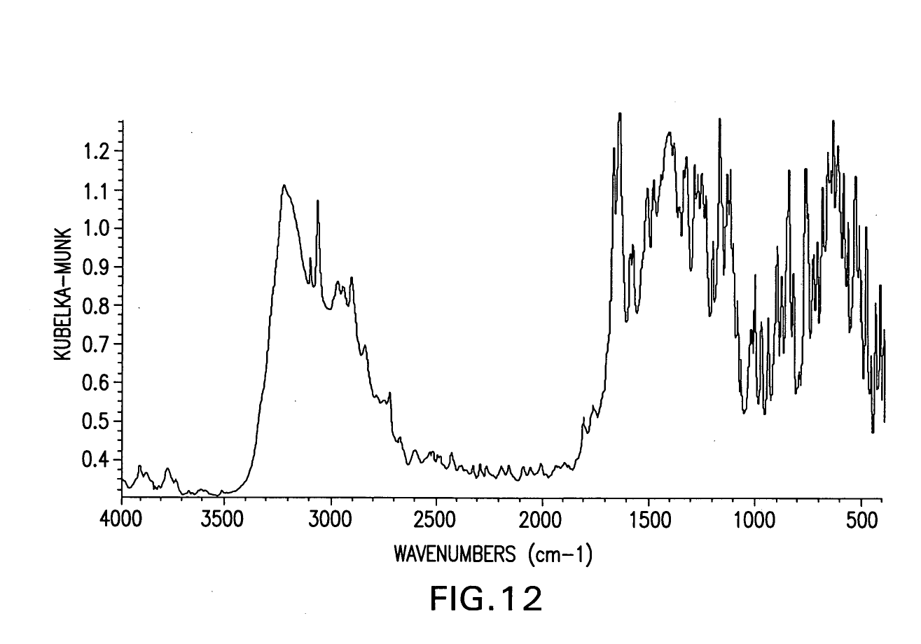

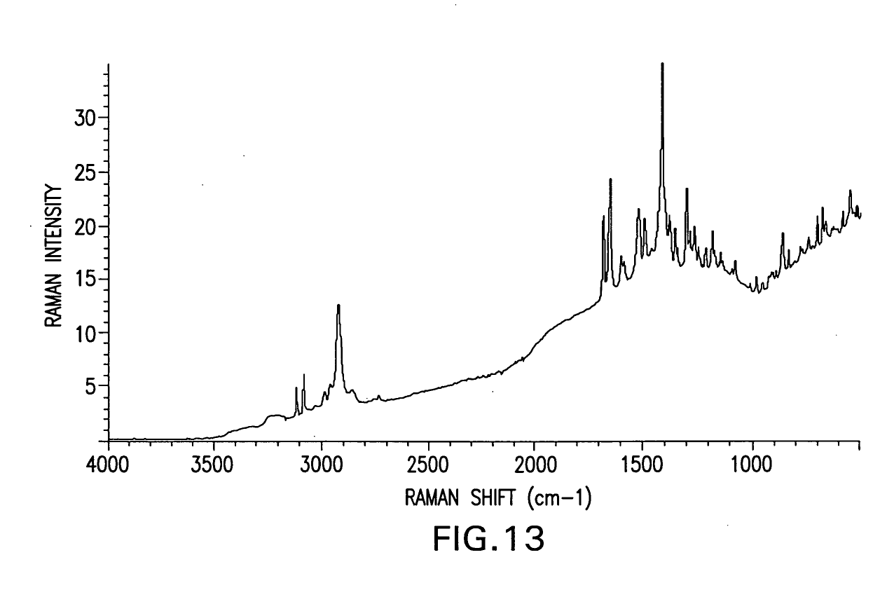

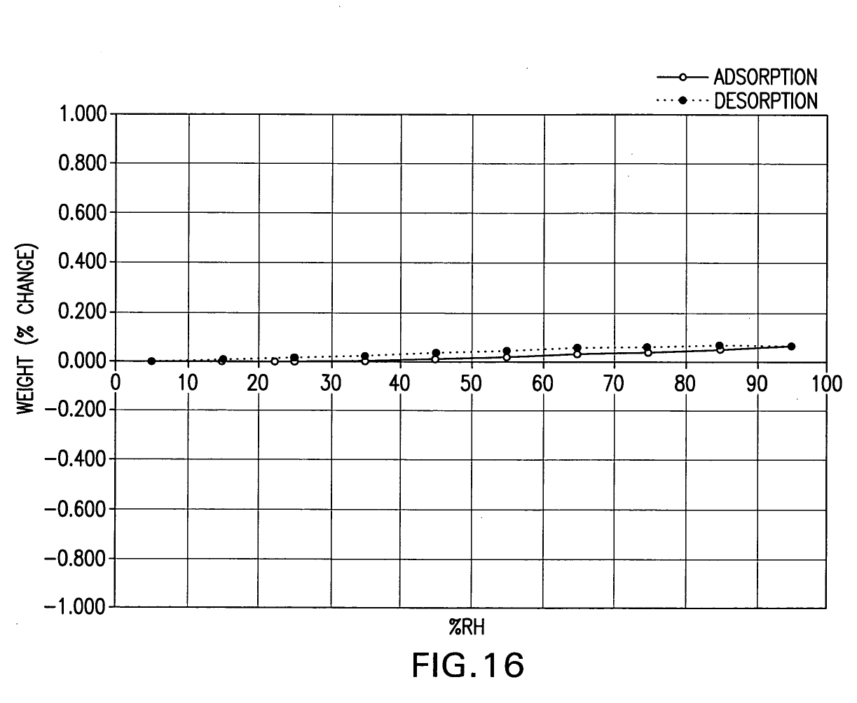

The XRPD pattern for Form C is shown in FIGS. 1 and 16. The IR and Raman data for Form C are summarized in Table 5 and 6 and plotted in FIGS. 12 and 13, respectively. The Form C IR spectrum has unique peaks at 3502, 3241(broad), 1684, 1525, 1402, 1293, 1140, 1017, 927(broad), 916, 896, 873, 810, 784, 775, 746, 728, 706, 680, 653, 580, and 513 cm−1 that are not found in the spectra of Forms A or E. The Form C Raman spectrum has unique peaks at 3083, 1684, 1291, 1221, 1179, and 867 cm−1 that are not found in the spectra of Forms A or E.

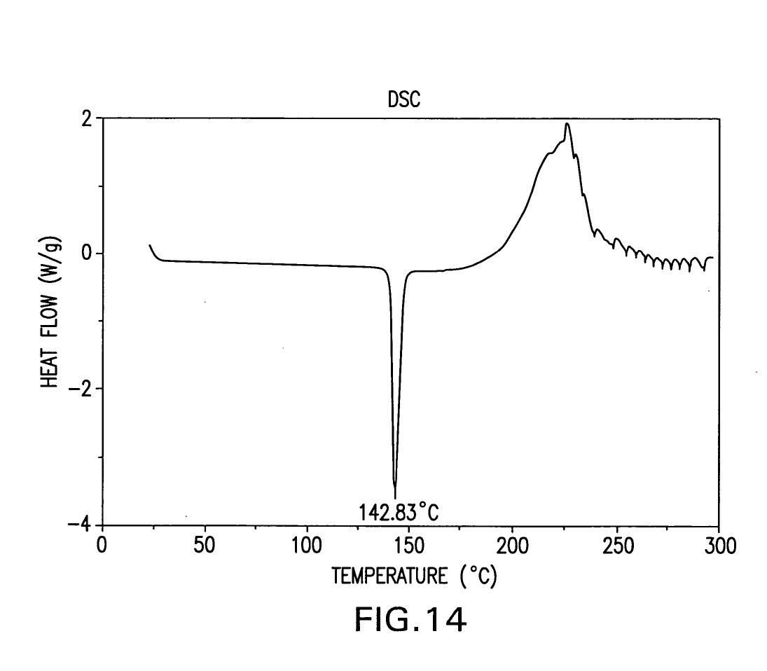

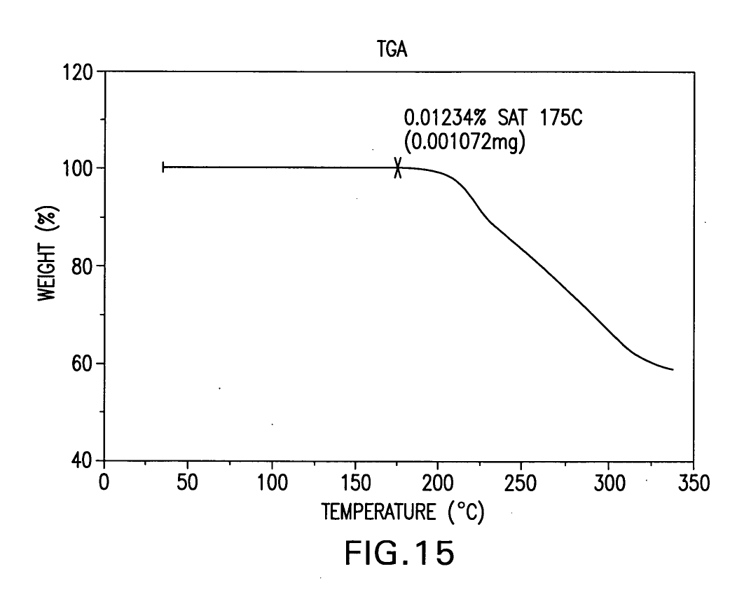

Thermal data for Form C are summarized in Tables 7 and 8 and plotted in FIGS. 14 and 15. The TG curve shows minimal volatile content at 175° C., indicating an unsolvated material. The DSC curve exhibited an endotherm at 142.8° C., which is attributed to melting based on the hot stage microscopy data. A sample of Form C generated in the polymorph screen displays an endotherm which is broader and slightly higher in temperature, possibly due to differences in particle size or crystallinity.

Moisture sorption/desorption data on Form C are summarized in Table 9 and plotted in FIG. 16. The material shows minimal weight loss or gain during the experiment. The XRPD pattern of the sample after the experiment was completed indicates that the material remained Form C. Form C appears to be a non-hygroscopic material based on the moisture sorption/desorption data.

Based on the characterization data, Form C appears to be an unsolvated, non-hygroscopic, crystalline material that melts at approximately 143° C.

3. Form E

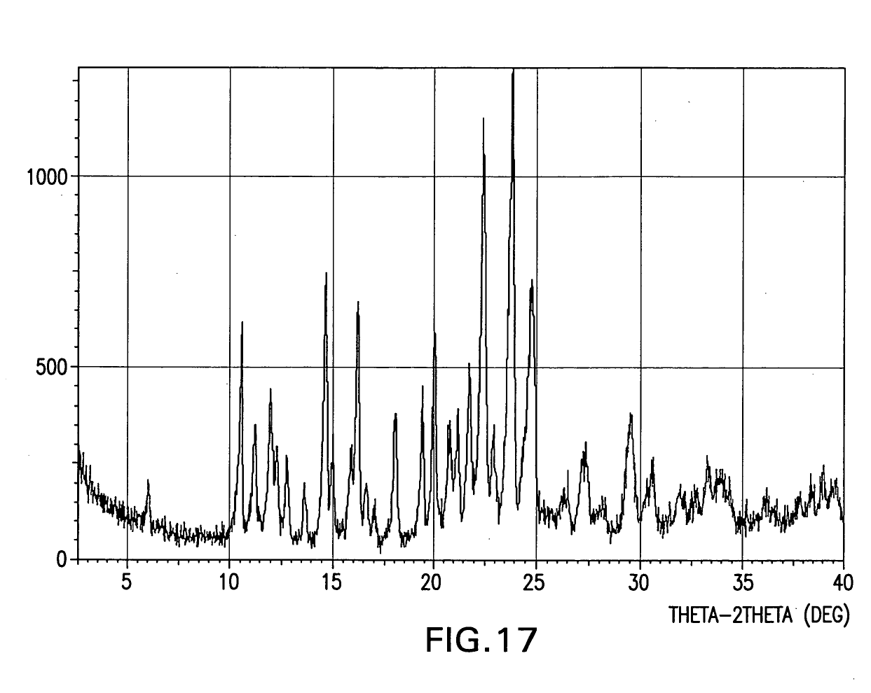

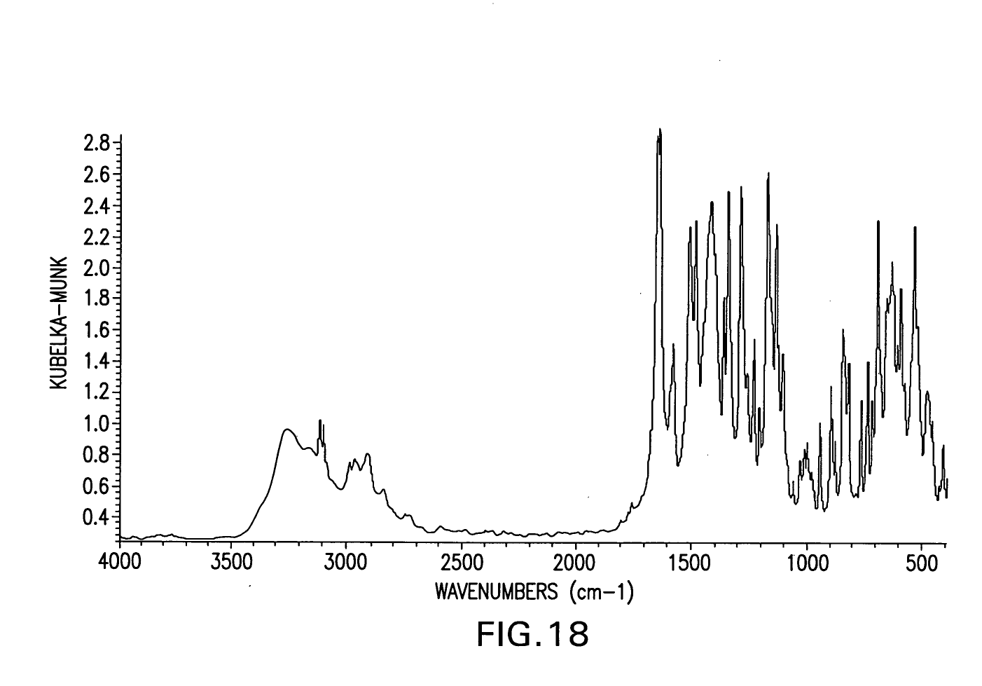

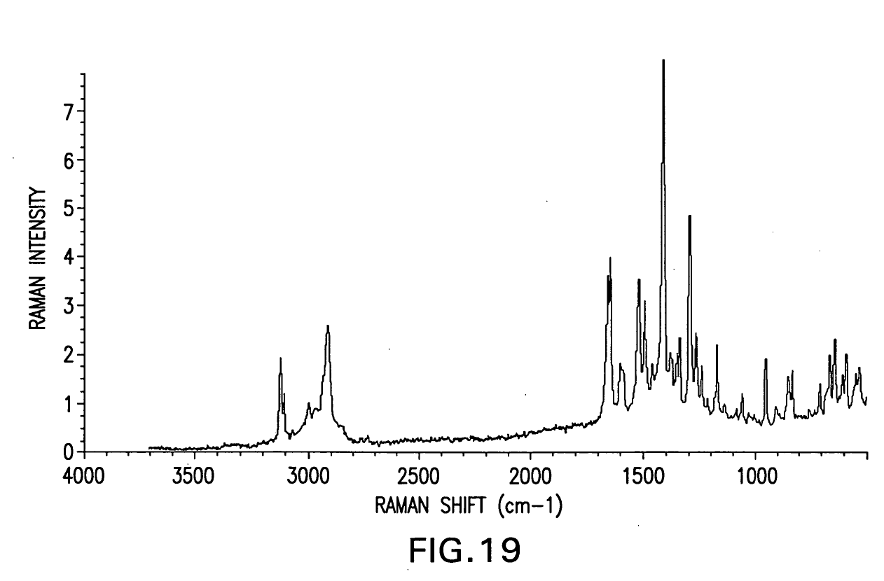

The XRPD pattern for Form E is shown in FIGS. 1 and 17. In the polymorph screen, Form E was most often produced from fast evaporation of solutions or antisolvent crystallizations. The IR and Raman data for Form E are summarized in Table 5 and 6 and plotted in FIGS. 18 and 19, respectively. The Form E IR spectrum has unique peaks at 3271(broad), 3129, 3005, 2943(broad), 1521, 1183, 1169, 1072, 1042, 911, 855, 752, and 645 cm−1 that are not found in the spectra of Forms C or E. The Form E Raman spectrum has unique peaks at 3131, 1418, 1066, and 645 cm−1 that are not found in the spectra of Forms C or E.

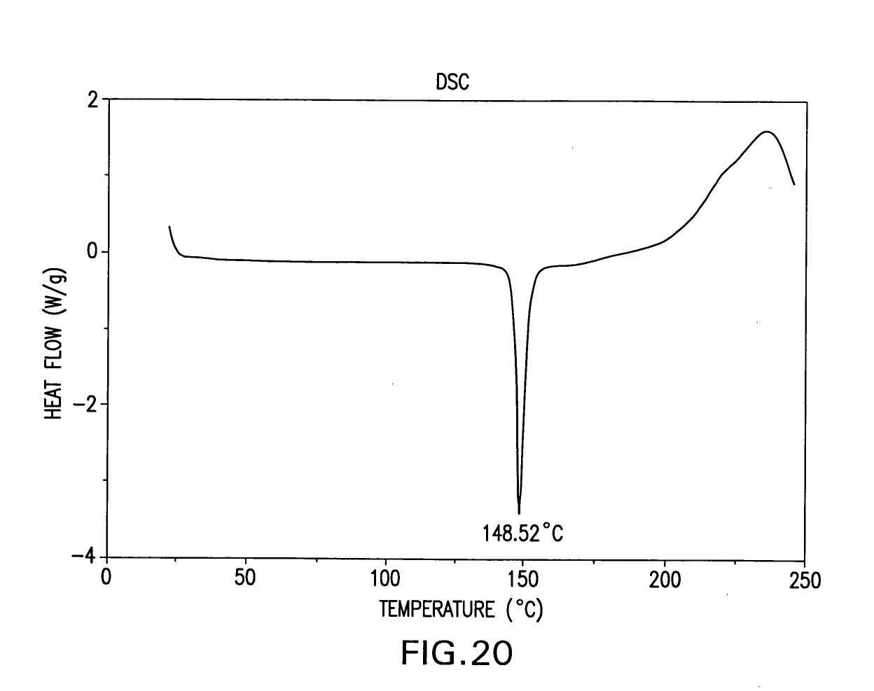

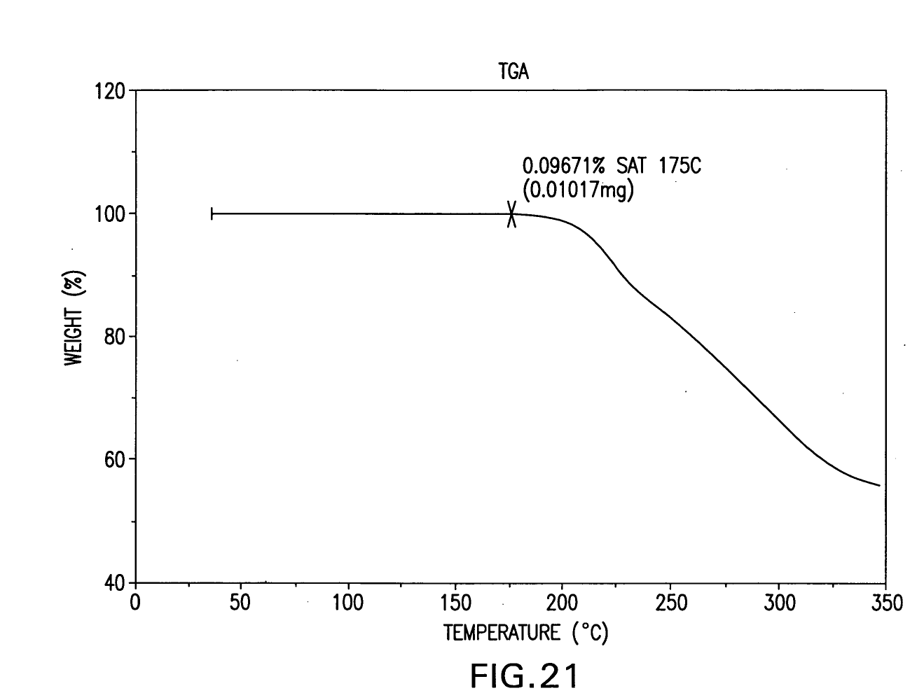

Thermal data for Form E are summarized in Tables 7 and 8 and plotted in FIGS. 20 and 21. The TG curve shows a total volatile content of 0.1% at 175° C., indicating an unsolvated material. The DSC curve exhibited an endotherm at 148.5° C., which is attributed to melting based on hot stage microscopy data (Table 8). A decomposition exotherm is observed above 200° C. A sample of Form E generated in the polymorph screen displays an endotherm which is broader and slightly lower in temperature.

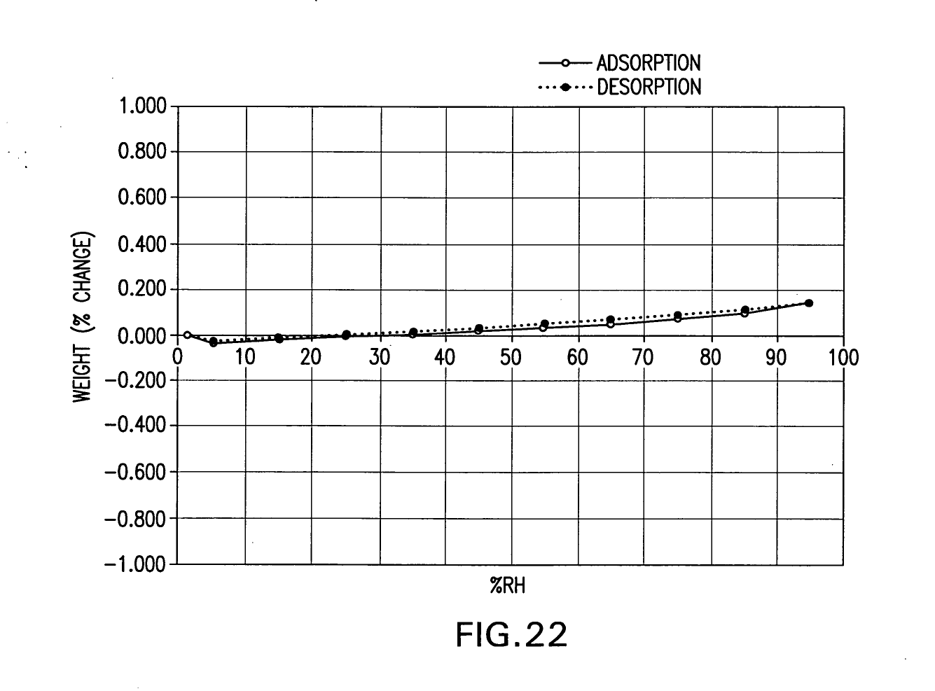

Moisture sorption/desorption data on Form E are summarized in Table 9 and plotted in FIG. 22. The material shows minimal weight loss or gain during the experiment (<0.2%). The XRPD pattern of the sample after the experiment was completed indicates that the material remained Form E. Based on the moisture sorption/desorption data, Form E appears to be a non-hygroscopic material.

Based on the characterization data, Form E appears to be an unsolvated, non-hygroscopic, crystalline material that melts at approximately 149° C.

4. Pattern D

Slow evaporation of N,N-dimethylformamide or toluene solutions of N-(2-acetyl-4,6-dimethylphenyl)-3-{[(3,4-dimethyl-5-isoxazolyl)amino]-sulfonyl}-2-thiophenecaboxamide sometimes produced material with a pattern different than that of Form A; this new pattern was initially designated as Pattern D. This pattern is similar to that of Form E, with additional peaks at 7.5 and 25.5° 2θ.

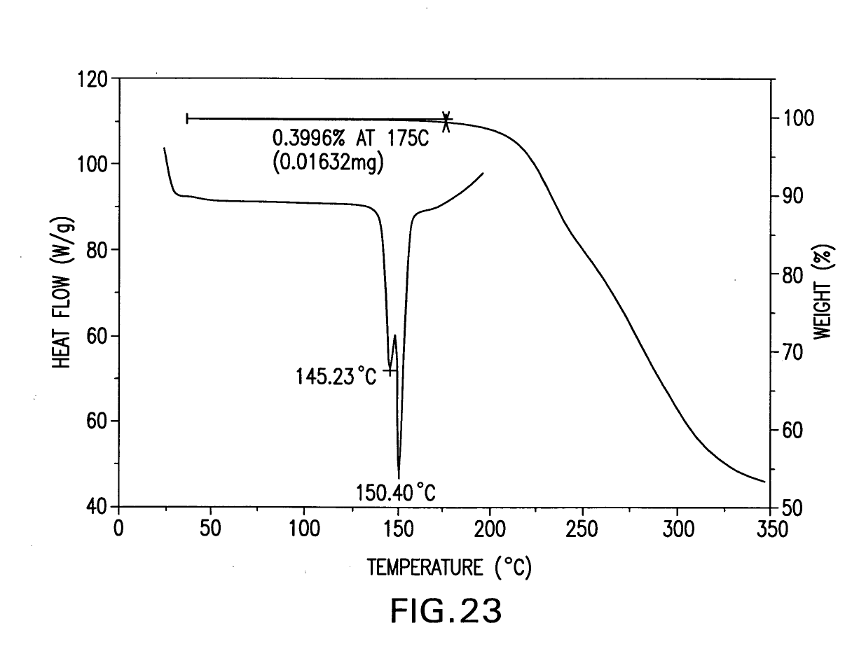

Thermal data for Pattern D material are summarized in Tables 7 and 8 and plotted in FIG. 23. The TG curve shows a total volatile content of 0.4% at 175° C., indicating an unsolvated material. The DSC curve exhibited two endotherms at 145.2° C. and 150.4. Hotstage microscopy confirmed that these events were due to the melting of separate portions of the sample, and not a melt and subsequent recrystallization. This indicates that the material consists of a mixture of two different crystalline forms, possibly Form E and Form A.

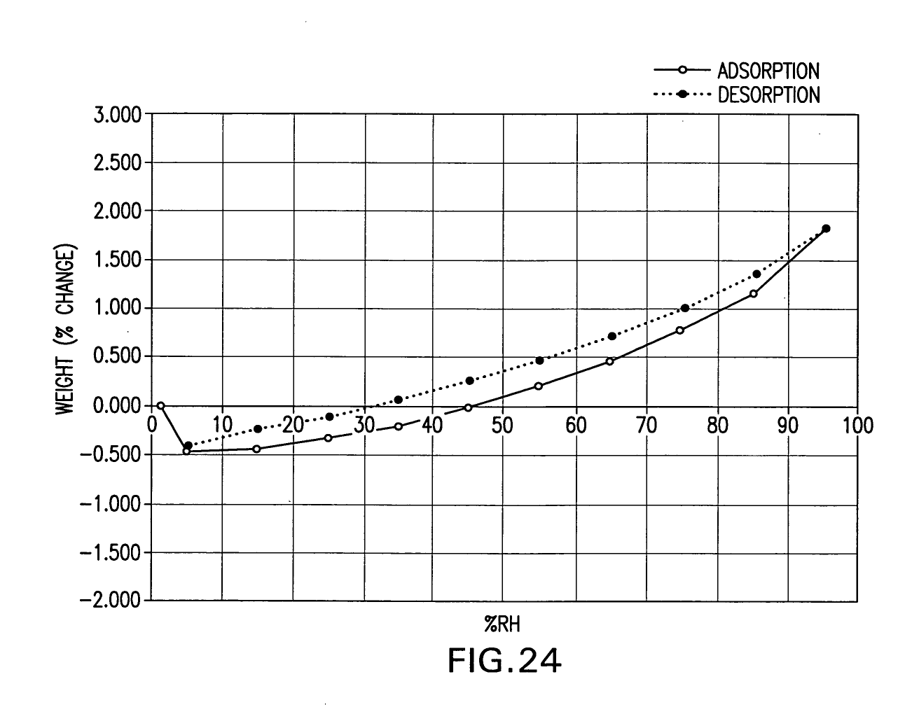

Moisture sorption/desorption data on Pattern D material are summarized in Table 9 and plotted in FIG. 24. The material shows a weight loss of 0.47% at 5% RH, and. a total gain of 1.82% at 95% RH. However, the XRPD of the initial material indicated the presence of amorphous material, which does absorb water under high RH conditions (see next section).

The experiments used to make the Pattern D material (slow evaporation of toluene solutions) were repeated, but were unsuccessful in preparing Pattern D material. Therefore, due to lack of material further experiments were not attempted. The thermal characterization data, however, indicate that the Pattern D material is not a new form but a mixture of forms.

5. Amorphous Material

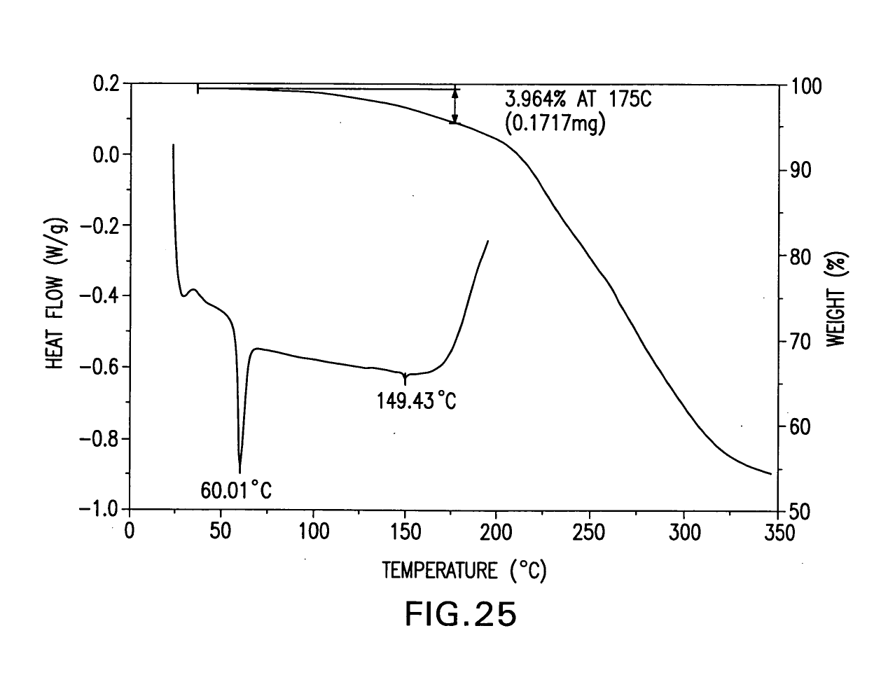

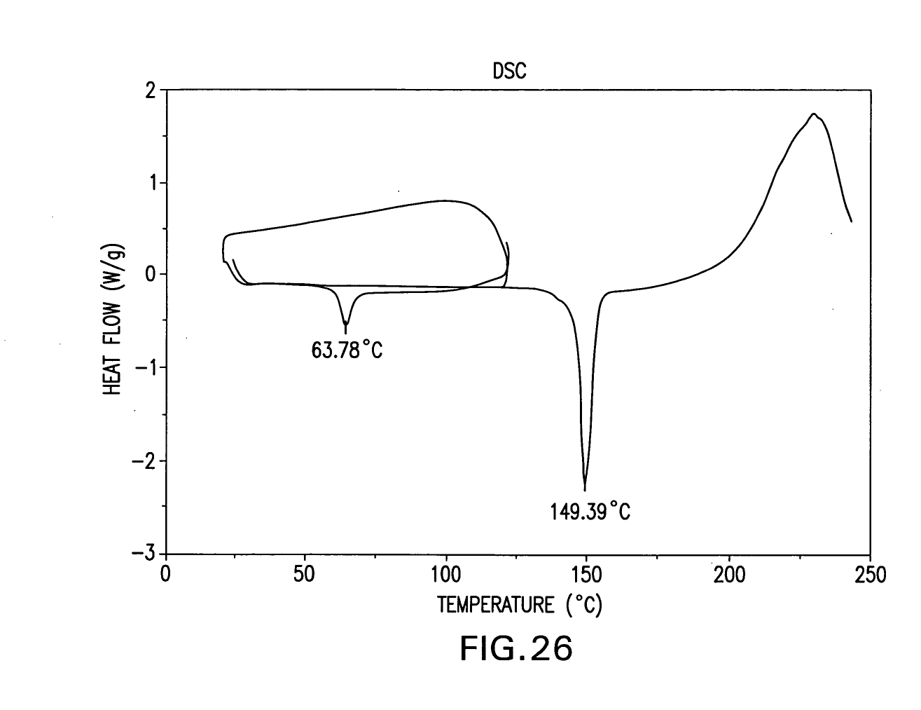

Amorphous N-(2-acetyl-4,6-dimethylphenyl)-3-{[(3,4-dimethyl-5-isoxazolyl)-amino]-sulfonyl}-2-thiophenecaboxamide was produced from quench cooling a melt and from methanol, as summarized in Table 3; the XRPD pattern is plotted in FIG. 1. The DSC and TG of the amorphous material formed from the slow evaporation of a THF solution are shown in FIG. 25. The TG curve shows that the material gradually loses weight as it is heated. The DSC curve displays an endotherm at approximately 60° C., and a small endotherm at 149° C. FIG. 26 shows attempts to measure the glass transition temperature (Tg) of the amorphous material using DSC temperature cycling experiments. Samples were cycled between 15 and 125° C., and also from −5 to 100° C., to remove any residual solvent and thus better detect the glass transition in the DSC trace. However, only the endotherm at approximately 50-60° C. was detected, followed by a melting endotherm at 149° C. Hotstage microsopy shows that the low temperature endotherm corresponds to the liquefication of the solid, and the formation of small crystals which melt upon heating.

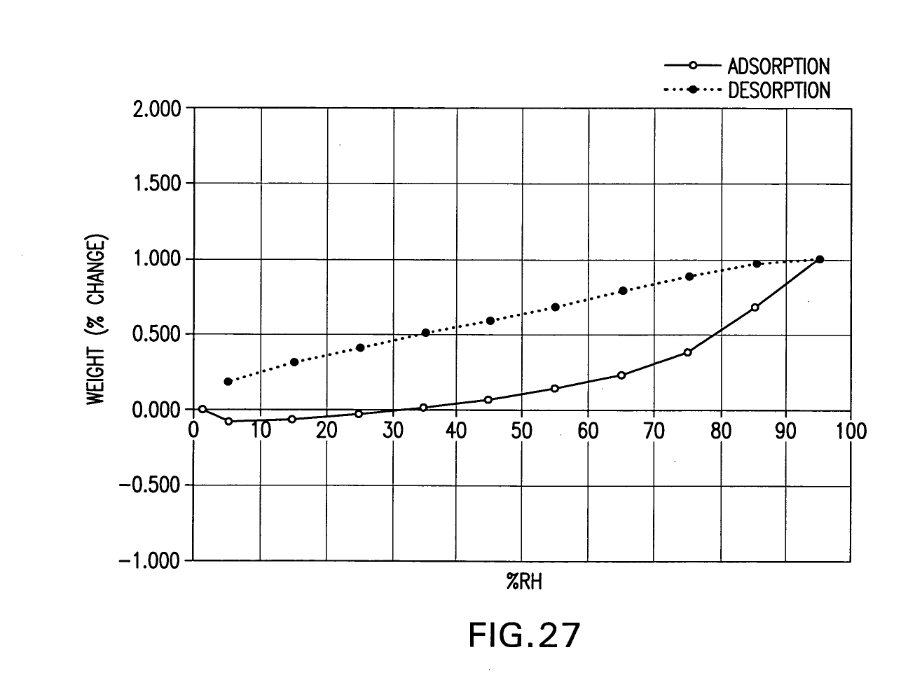

Moisture sorption/desorption data for amorphous N-(2-acetyl-4,6-dimethylphenyl)-3-{[(3,4-dimethyl-5-isoxazolyl)amino]-sulfonyl}-2-thiophenecaboxamide was also collected, as summarized in Table 9 and plotted in FIG. 27. The material gains 0.98% water at 95% RH and loses most of this gain upon lowering the RH. XRPD data collected on the sample after the moisture balance run shows that the material remained amorphous.

Based on these data, the amorphous material is relatively stable and does not recrystallize upon exposure to high RH.

Stress Studies

N-(2-acetyl-4,6-dimethylphenyl)-3-{[(3,4-dimethyl-5-isoxazolyl)amino]-sulfonyl}-2-thiophenecaboxamide Form A was stressed under various conditions, as summarized in Table 10. The material remained Form A in all cases. Amorphous N-(2-acetyl-4,6-dimethylphenyl)-3-{[(3,4-dimethyl-5-isoxazolyl)amino]-sulfonyl}-2-thiophenecaboxamide was also stressed under various conditions. After four days at 70° C., the amorphous material had converted to a mixture of Forms E and C. A sample of amorphous material was found to have crystallized after approx. 3 months at ambient conditions to Forms C and E.

TABLE 10
Stress Studies

| Initial | | XRPD |
|---|---|---|
| Form | Conditions | Pattern |
| A | 22% RH, 3 d | A |
| A | 58% RH, 3 d | A |
| A | 75% RH, 3 d | A |
| A | 93% RH, 3 d | A |
| A | 40° C., 11 d | A |
| amorphous | 40° C., 36 d | amorphous |
| amorphous | 70° C., 4 d | C + E |
| amorphous | ambient, 92 d | C + E |

Grinding Experiments

Form A material was ground for up to 5 minutes using a mortar and pestle, as summarized in Table 17. Form A loses some crystallinity upon grinding, as shown in FIG. 17. Pure amorphous material is not obtained under these conditions, but some amorphous material appears to be present. However, more energetic processing conditions (such as milling or micronization) may increase the amount of amorphous material produced.

One sample from the polymorph screen was ground for two minutes in an attempt to remove the effects of preferred orientation. The pattern of the resulting material shows both forms C and E are present. A sample of Form C was also ground for 2 minutes, and the resulting material remained Form C.

TABLE 11
Grinding Experiments

| Initial | Grind | XRPD |
|---|---|---|
| Form^{a} | Time | Pattern^{b} |
| A | 30 s | A |
| A | 1 min | A, SS |
| A | 2 min | A |
| A | 5 min | A |
| C | 2 min | C |
| A(PO) | 2 min | C + E |

^{a}PO = preferred orientation
^{b}SS = small sample

Interconversion Experiments

Interconversion studies were performed in 1:1 methanol:water, toluene, and ethanol using Forms A, C, and E, and the data are summarized in Table 12. To confirm that Form C is one form and not a mixture of forms, a sample of Form C was slurried in ethanol for 16 days. It remained the same form, indicating that no interconversion took place and it is indeed a distinct form and not a mixture of forms.

Experiments using Form A versus Form C in these solvents yielded mixtures of the two forms, most likely due to the low solubility of both forms, necessitating longer equilibration times. Slurries of Forms E and C together yielded solids that are mixtures of Forms A and C or Form A alone, indicating that Form A had nucleated during the experiment.

TABLE 12
Interconversion Studies for N-(2-acetyl-4,6-dimethylphenyl)-3-{[(3,4-
dimethyl-5-isoxazolyl)amino]-sulfonyl}-2-thiophenecaboxamide

| | | Time | XRPD |
|---|---|---|---|
| Solvent | Forms^{a} | (days) | Results^{b} |
| ethanol | A vs. E | 20 | A + E(min) |
| | A vs. E | 27 | A + pk 4° |
| | A vs. C | 12 | A |
| | C | 16 | C |
| | C vs. E | 44 | A |
| 1:1 | A vs. E | 20 | A |
| methanol:water | A vs. E | 27 | A |
| | A vs. C | 12 | A + C, SS |
| toluene RT | A vs. E | 20 | A, SS |
| | A vs. E | 27 | A |
| | A vs. C | 12 | A + C |
| toluene, 45° C. | C vs. E | 5 | A + C |
| | A vs. E | 5 | A(PO) |
| | A vs. C | 5 | A + C |

^{a}lots used: Form A; Form C; Form E,
^{b}SS = small sample; min = minor

D. Process for the Preparation of Forms A, C and E

Based on the crystallization data, Form A appears to be the favored form of N-(2-acetyl-4,8-dimethylphenyl)-3-{[(3,4dimethyl-5-isoxazolyl)arninolsulfonyl}-2-thiophenecarboxamide at elevated temperatures and when Form A seed crystals are present.

If a saturated solution of this compound is allowed to crystallize at low temperatures (5° C.), without the presence of Form A seed crystals, then Form E is produced. Form E appears to persist and does not readily convert to Form A.under these conditions.

Experiments conducted at all temperatures which involved Form A or C seed crystals resulted in the crystallization of Form A, however, the formation of Form A was slower at lower temperatures.

In certain embodiments, the process for crystallization of N-(2-acetyl-4,8-dimethylphenyl)-3-{[(3,4dimethyl-5-isoxazolyl)arninolsulfonyl}-2-thiophenecarboxamide provided herein produces greater than 70% polymorph A. In one embodiment, the process yields about 70%, 75%, 80%, 85%, 90%, 95%, 96%, 97%, 98%, 99% or 100% polymorph A.

In certain embodiments, the process for crystallization of N-(2-acetyl-4,8-dimethylphenyl)-3-{[(3,4dimethyl-5-isoxazolyl)arninolsulfonyl}-2-thiophenecarboxamide provided herein produces greater than 70% polymorph C. In one embodiment, the process yields about 70%, 75%, 80%, 85%, 90%, 95%, 96%, 97%, 98%, 99% or 100% polymorph C.

In certain embodiments, the process for crystallization of N-(2-acetyl-4,8-dimethylphenyl)-3-{[(3,4dimethyl-5-isoxazolyl)arninolsulfonyl}-2-thiophenecarboxamide provided herein produces greater than 70% polymorph E. In one embodiment, the process yields about 70%, 75%, 80%, 85%, 90%, 95%, 96%, 97%, 98%, 99% or 100% polymorph E.

E. Formulation and Administration of the Compositions

Formulations of the sulfonamides are provided herein. The formulations are compositions designed for administration of the polymorphs provided herein. The compositions are suitable for oral and parental administerations. Such compositions include solutions, suspensions, tablets, dispersible tablets, pills, capsules, powders, sustained release formulations and any other suitable formation. In one embodiment, compositions will take the form of a pill or tablet. Methods for manufacture of tablets, capsules and other such formulations are known to those of skill in the art (see, e.g., Ansel, H. C (1985) Introduction to Pharmaceutical Dosage Forms,, 4th Edition, pp. 126-163).

In the formulations provided herein, effective concentrations of a single polymorph is mixed with a suitable pharmaceutical carrier or vehicle. The concentrations of the polymorphs in the formulations are effective for delivery of an amount, upon administration, that ameliorates the symptoms of the endothelin-mediated disease. In one embodiment, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. Liposomal suspensions, including tissue-targeted liposomes, may also be suitable as pharmaceutically acceptable carriers. These may be prepared according to methods known to those skilled in the art. For example, liposome formulations may be prepared as described in U.S. Pat. No. 4,522,811.

The active compound as a single polymorph, is included in the pharmaceutically acceptable carrier in an amount sufficient to exert a therapeutically useful effect in the absence of undesirable side effects on the patient treated. The therapeutically effective concentration may be determined empirically by testing the compounds in known in vitro and in vivo systems (see, e.g., U.S. Pat. No. 5,114,918 to Ishikawa et al.; EP A1 0 436 189 to BANYU PHARMACEUTICAL CO., LTD (Oct. 7, 1991); Borges et al. (1989) Eur. J. Pharm. 165: 223-230; Filep et al. (1991) Biochem. Bioohvs. Res. Commun. 177: 171-176) and then extrapolated therefrom for dosages for humans.

The concentration of active compound single polymorph in the drug composition will depend on absorption, inactivation and excretion rates of the active compound, the physicochemical characteristics of the compound, the dosage schedule, and amount administered as well as other factors known to those of skill in the art. For example, the amount that is delivered is sufficient to treat the symptoms of hypertension. The effective amounts for treating endothelin-mediated disorders are expected to be higher than the amount of the sulfonamide compound that would be administered for treating bacterial infections.

In one embodiment, a therapeutically effective dosage should produce a serum concentration of active ingredient of from about 0.1 ng/ml to about 50100 pg/ml. The pharmaceutical compositions should provide a dosage of from about 0.001 mg to about 2000 mg of compound per kilogram of body weight per day. In one embodiment, pharmaceutical dosage unit forms are prepared to provide from about 1 mg to about 1000 mg and in another embodiment may from about 10 to about 500 mg of the active ingredient or a combination of active ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Pharmaceutically acceptable derivatives include acids, salts, esters,' hydrates, solvates and prodrug forms. The derivative is selected to be a more stable form than the corresponding neutral compound.

Thus, effective concentrations or amounts of a single polymorph provided, herein or pharmaceutically acceptable derivatives thereof are mixed with a suitable pharmaceutical carrier or vehicle for systemic, topical or local administration to form pharmaceutical compositions. Compounds are included in an amount effective for ameliorating or treating the endothelin-mediated disorder for which treatment is contemplated. The concentration of active compound in the composition will depend on absorption, inactivation, excretion rates of the active compound, the dosage schedule, amount administered, particular formulation as well as other factors known to those of skill in the art.

The compositions are intended to be administered by an suitable route, which includes orally, parenterally, rectally and topically and locally depending upon the disorder being treated. For example, for treatment of ophthalmic disorders, such as glaucoma, formulation for intraocular and also intravitreal injection is contemplated. In one embodiment, capsules and tablets are used for oral administration. Reconstitution of a lyophilized powder, prepared as described herein, maybe used for parental administration. The compounds in liquid, semi-liquid or solid form and are formulated in a manner suitable for each route of administration. Modes of administration include parenteral and oral modes of administration.

Solutions or suspensions used for parenteral, intradermal, subcutaneous, or topical application can include any of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose, Parentaral preparations can be enclosed in ampules, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as tween, or dissolution in aqueous sodium bicarbonate. Derivatives of the compounds, such as prodrugs of the compounds may also be used in formulating effective pharmaceutical compositions.

Upon mixing or addition of the sodium salt of the sulfonamide compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The formulations are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds, particularly the pharmaceutically acceptable salts, such as the sodium salts, thereof. The pharmaceutically therapeutically active compounds and derivatives thereof are formulated and administered in unit dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes individually packaged tablet or capsule. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pint or gallons. Hence, multiple dose form is a multiple of unit-doses which are not segregated in packaging.

The composition can contain along with the active ingredient: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polvinylpyrrolidine, celluloses and derivatives thereof, povidone, crospovidones and other such binders known to those of skill in the art. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, for example, acetate, sodium citrate, cyclodextrine derivatives, sorbitan monolaurate, triethanolamine sodium acetate, triethanolamine oleate, and other such agents. Actual methods of preparing such dosage forms are known, or will be apparent, to those skilled in this art; for example, see Remington's Pharmaceutical Sciences, Mack Publishing Company, Easton, Pa., 15th Edition, 1975. The composition or formulation to be administered will, in any event, contain a quantity of the active compound in an amount sufficient to alleviate the symptoms of the treated subject.

Dosage forms or compositions containing active ingredient in the range of 0.005% to 100% with the balance made up from non-toxic carrier may be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of .these formulations are known to those skilled in the art. In an embodiment, the contemplated compositions may contain 0.001%-100% active ingredient, in another embodiment 0.1-85%, in another embodiment 75-95%.

The salts, such as sodium salts, of the active compounds may be prepared with carriers that protect the compound against rapid elimination from the body, such as time release formulations or coatings.

The formulations may be include other active compounds to obtain desired combinations of properties. The compounds of formula I, or a pharmaceutically acceptable salts and derivatives thereof as described herein, may also be advantageously administered for therapeutic or prophylactic purposes together with another pharmacological agent known in the general art to be of value in treating one or more of the diseases or medical conditions referred to hereinabove, such as beta-adrenergic blocker (for example atenolol), a calcium channel blocker (for example nifedipine), an angiotensin converting enzyme (ACE) inhibitor (for example lisinopril), a diuretic (for example furosemide or hydrochiorothiazide), an endothelin converting enzyme (ECE) inhibitor (for example phosphoramidon), a neutral endopeptidase (NEP) inhibitor, an HMGCoA reductase inhibitor, a nitric oxide donor, an anti-oxidant, a vasodilator, a dopamine agonist, a neuroprotective agent, a steroid, a beta-agonist, an anti-coagulant, or a thrombolytic agent. It is to be understood that such combination therapy constitutes a further aspect of the compositions and methods of treatment provided herein.

1. Formulations for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms such as capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent. Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumia hydrate. Sweetening agents include sucrose, lactose, mannitol and artificial sweetening agents such as sodium cyclamate and saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylceilulose, polyethylene glycol 4000 and cellulose acetate phthalate.

If oral administration is desired, the salt of the compound could be provided in a composition. that protects. it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. For example, if the compound is used for treating asthma or hypertension, it may be used with other bronchodilators and antihypertensive agents, respectively. The active ingredient is a compound or salt thereof as described herein. Higher concentrations, up to about 98% by weight of the active ingredient may be included.

Pharmaceutically acceptable carriers included in tablets are binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets are compressed tablets to which different layers of pharmaceutically acceptable substances are applied. Film-coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar-coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substance used in effervescent granules, to be reconstituted into a liquid oral dosage form, include organic adds and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions Include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents Include sucrose, syrups, glycerin and artificial sweetening agents such as sodium cyclamate and saccharin. Wetting agents include propylene glycol monostearate, sorbitan,monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, In for example propylene carbonate, vegetable oils or triglycerides, are encapsulated in a gelatin capsule. Such solutions, and the preparation and encapsulation thereof, are disclosed in U.S. Pat. Nos 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g. water, to be easily measured for administration.

Alternatively, liquid or semi-solid oral formulations may be prepared by dissolving or dispersing the active compound or salt in vegetable oils, glycols, triglycerides, propylene glycol asters (e.g. propylene carbonate) and other such carriers, and encapsulating these solutions or suspensions in, hard or soft gelatin capsule shells. Other useful formulations include those set forth in U.S. Pat. Nos. Re 28,819 and 4,358,603.

In one embodiment, the formulations are solid dosage forms, such as capsules or tablets. In another embodiment, the formulations are solid dosage forms, such as capsules or tablets, containing 10-100%, in another embodiment 50-95%, in another embodiment 75-85%, in another embodiment 80-85%, by weight of a single polymorph provided herein; 0-25%, in another embodiment 8-15%, of a diluent or a binder, such as lactose or microcrystalline cellulose; about 0 to 10%, in another embodiment about 0-7%, of a disintegrant, such as a modified starch or cellulose polymer, particularly a cross-linked sodium carboxymethyl cellulose, such as crosscarmellose sodium (Crosscarmellose sodium NF is available commercially under the name AC-DI-SOS, FMC Corporation, Philadelphia, Pa.) or sodium starch glycolate; and 0-2% of a lubricant, such a magnesium stearate, talc and calcium stearate. The disintegrant, such as crosscarmellose sodium or sodium starch glycolate, provides for rapid break-up of the cellulosic matrix for immediate release of active agent following dissolution of coating polymer. In all embodiments, the precise amount of active ingredient and auxilliary ingredients can be determined empirically and is a function of the route of administration and the disorder that is treated.

In an exemplary embodiment, the formulations are capsules containing about 50%-100%, in another embodiment about 70-90%, in another embodiment about 80-90%, in another embodiment about 83% of a single polymorph provided herein; about 0-15%, in another embodiment about 11% of a diluent or a binder, such as lactose or microcrystalline cellulose; about 0-10%, in another embodiment about 5% of a disintegrant, such as crosscarmellose sodium or sodium starch glycolate; and about 0 to 5%, in another embodiment about 1% of a lubricant, such as magnesium stearate. Solid forms for administration as tablets are also contemplated herein.

In an exemplary embodiment, the formulations are capsules containing 83% of one a single polymorph provided herein; 11% of microcrystalline cellulose; 5% of a disintegrant, such as Crosscarmellose sodium or sodium starch glycolate; and 1% of magnesium stearate.

The above embodiments may also be formulated in the form of a tablet, which may optionally be coated. Tablets will contain the compositions described herein.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

2. Sustained Release Dosage Form

Polymorphs provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

In certain embodiments, the polymorph or mixture of polymorphs may be administered using intravenous infusion, an implantable osmotic pump, a transdermal patch, liposomes, or other modes of administration. In one embodiment, a pump may be used (see, Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity of the therapeutic target, i.e., thus requiring only a fraction of the systemic dose (see, e.g., Goodson, Medical Applications of Controlled Release, vol. 2, pp. 115-138 (1984). In some embodiments, a controlled release device is introduced into a subject in proximity of the site of inappropriate immune activation or a tumor. Other controlled release systems are discussed in the review by Langer (Science 249:1527-1533 (1990). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

3. Injectables, Solutions and Emulsions

Parenteral administration, generally characterized by injection, either subcutaneously, intramuscularly or intravenously is also contemplated herein. Injectables can lx: prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolarnine oleate and cyclodextrins. Implantation of a slow-release or sustained-release system, such that a constant level of dosage is maintained (see, e.g., U.S. Pat. No. 3,710,795) is also contemplated herein. The percentage of active compound contained in. such parenteral compositions is highly dependent on the specific nature thereof, as well as the activity of the compound and the needs of the subject.

Parenteral administration of the formulations includes intravenous, subcutaneous and intramuscular administrations. Preparations for parenteral administration include sterile solutions ready for injection, sterile dry soluble products, such as the lyophilized powders described herein, ready to be combined with a solvent just prior to use, including hypodermic tablets, sterile suspensions ready for injection, sterile dry Insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkcnium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyi mathylceilulose and polyvinylpyrrolidone. Emulsifying agents include Polysorbate 80 (Tween 80'°). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration must be sterile, as is know and practiced in the art.

Illustratively, intravenous or intraarterial infusion of a sterile aqueous solution containing an active compound is an effective mode of administration. Another embodiment is a sterile aqueous or oily solution or suspension containing an active material Injected as necessary to produce the desired pharmacological effect.

Injectables are designed for local and systemic administration. In one embodiment a therapeutically effective dosage is formulated to contain a concentration of at least about 0.1% w/w up to about 90% w/w or more, in another embodiment more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols. or by extrapolation from in vivo or in vitro test data. It is to be noted that concentrations and dosage values may also vary with the age of the individual treated. It is to be further understood that for any particular subject, specific dosage regimens should be adjusted overtime according to the individual need and the professional judgment of the person administering or supervising the administration of the formulations, and that the concentration ranges set forth herein are exemplary only and are not intended to limit the scope or practice of the claimed formulations.

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

4. Lyophilized Powders

Of interest herein are lyophilized powders, which can be reconstituted for administration as solutions, emulsions and other mixtures. They may also be reconsitituted and formulated as solids or gels.

Formulation of sulfonamide sodium salts as a sterile, lyophilized powder are provided herein. These powders were found to have increased stability relative to formulations of the neutral sulfonamides.

The sterile, lyophilized powder is prepared by dissolving the single polymorph in a sodium phosphate buffer solution containing dextrose or other suitable excipient. Subsequent sterile filtration of the solution followed by lyophilization under standard conditions known to those of skill in the art provides the desired formulation. Briefly, in one embodiment the lyophilized powder is prepared by dissolving dextrose, sorbital, fructose, corn syrup, xylitol, glycerin, glucose, sucrose or other suitable agent, about 1-20%, in another embodiment about 5 to 15%, in a suitable buffer, such as citrate, sodium or potassium phosphate or other such buffer known to those of skill in the art at, such as, about neutral pH. Then, a selected salt, for example the sodium salt of the sulfonamide (about 1 gm of the salt per 10-100 gms of the buffer solution, in one embodiment about 1 gm/30 gms), is added to the resulting mixture, in one embodiment above room temperature, in another embodiment about 30-35° C., and stirred until it dissolves. The resulting mixture is diluted by adding more buffer (so that the resulting concentration of the salt decreases by about 10-50%, in one embodiment about 15-25%). The resulting mixture is sterile filtered or treated to remove particulates and to insure sterility, and apportioned into vials for lyophilization. Each vial will contain a single dosage (in one embodiment 100-500 mg, in another embodiment 250 mg) or multiple dosages of the sulfonamide salt. The lyophilized powder can be stored under appropriate conditions, such as at about 4° C. to room temperature. Details of an exemplary procedure are set forth in the Examples.

Reconstitution of this lyophilized powder with water for injection provides a formulation for use in parenteral administration of compounds. In one embodiment reconstitution of about 1-50 mg, in another embodiment 5-35, in another embodiment about 9-30 is added per ml of sterile water or other suitable carrier. The precise amount depends upon the indication treated and selected compound. Such amount can be empirically determined.

In one embodiment, the formulations contain lyophilized solids containing a single polymorph as provided herein, and also contain one or more of the following a buffer, such as sodium or potassium phosphate, or citrate;

a solubilizing agent, such as LABRASOL, DMSO, bis(trimethylsiiyl)acetamide, ethanol, propyleneglycol (PG), or polyvinylpyrrolidine (PVP); and

a sugar, such as sorbitol or dextrose.

In other embodiments, the formulations contain a single polymorph provided herein; a buffer, such as sodium or potassium phosphate, or citrate; and a sugar, such as sorbitol or dextrose.

In other embodiments, the formulations contain a single polymorph provided herein; a sodium phosphate buffer; and dextrose.

5. Topical Administration

Topical mixtures are prepared as described for the local and systemic administration. The resulting mixture may be a solution, suspension, emulsions or the like and are formulated as creams, gets, ointments, emulsions, solutions, elixirs, lotions, suspensions, tinctures, pastes, foams, aerosols, irrigations, sprays, suppositories, bandages, dermal patches or any other formulations suitable for topical administration.

The polymorphs may be formulated as aerosols for topical application, such as by inhalation (see, e.g., U.S. Pat. Nos. 4,044,126, 4,414,209, and 4,364,923, which describe aerosols for delivery of a steroid useful for treatment inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will typically diameters of less than 50 microns, in another case less than 10 microns.

The polymorphs may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

6. Formulations for Other Routes of Administration

Depending upon the condition treated other routes of administration, such as topical application, transdermal patches, an rectal administration are also contemplated herein.

For example, pharmaceutical dosage forms for rectal administration are rectal suppositories, capsules and tablets for systemic effect. Recta suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax, (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. In one embodiment, the weight of a rectal suppository is about 2 to 3 gm.

Tablets and capsules for rectal administration are manufactured using the same pharmaceutically acceptable substance and by the same methods as for formulations for oral administration.

7. Articles of Manufacture

The polymorphs may be packaged as articles of manufacture containing packaging material, a polymorph as provided herein, which is effective for antagonizing the effects of endothelin, ameliorating the symptoms of an endothelin-mediated disorder, or inhibiting binding of an endothelin peptide to an ET receptor with an IC₅₀ of less than about 10 p.m., within the packaging material, and a label that indicates that the polymorph is used for antagonizing the effects of endothelin, treating endothelin-mediated disorders or inhibiting the binding of an endothelin peptide to an ET receptor.

The articles of manufacture provided herein contain packaging materials. Packaging materials for use in packaging pharmaceutical products are well known to those of skill in the art. See, eg., U.S. Pat. Nos. 5,323,907, 5,052,558 and 5,033,352. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety treatments for any disorder in which endothelin receptors are implicated as mediators or contributors to the symptoms or cause.

F. Methods of Use of the Polymorphs of N-(2-acetyl-4,6-dlmethyiphenyl)-3-{[(3,4dimethyi-5-isoxazolyl)-aminosulfonyl}-2-thlophenecarboxamide

N-(2-acetyl-4,6-dimethylphenyt)-3-{[(3,4dimethyl-5-isoxazolyl)aminolsulfonyl}-2-thiophenecarboxamide in polymorph Forms A, C and E are useful in the treatment of endothelin-mediated diseases. These treatments encompass administering to a subject an effective amount of Forms A, C or E, wherein the effective amount is sufficient to ameliorate one or more of the symptoms of the disease.

Polymorphs A, C and E are effective for the treatment of hypertension, cardiovascular diseases, cardiac diseases including myocardial infarction, pulmonary hypertension, neonatal pulmonary hypertension, erythropoletin hypertension, respiratory diseases, and inflammatory diseases, including asthma, bronchoconstriction, ophthalmologic diseases including glaucoma and inadequate retinal perfusion, gastroenteric diseases, renal failure, endotoxin shock, menstrual disorders, obstetric conditions, wounds, laminitis, erectile dysfunction, menopause, osteoporosis and metabolic bone disorders, climacteric disorders including hot flushes, abnormal clotting patterns, urogenital discomfort and increased incidence of cardiovascular disease and other disorders associated with the reduction in ovarian function in middle-aged women, pre-eclampsia, control and management of labor during pregnancy, nitric oxide attenuated disorders, anaphylactic shock, hemorrhagic shock and immunosuppressant-mediated renal vasoconstriction.

Polymorphs A, C and E are also useful for inhibiting the binding of an endothelin peptide to an endothelin_{A} (ET_{A}) or endothelin_{B} (ET_{B}) receptor. This inhibiting encompasses contacting the receptor with any of the polymorphs A, C or E, or a pharmaceutially acceptable derivative thereof, wherein the contacting is effected prior to, simultaneously with or subsequent to contacting the receptor with the endothelia peptide.

Polymorphs A, C and E are useful for altering endothelin receptor-mediated activity, This altering encompasses contacting an endothelin receptor with any of the polymorphs A, C or E.

G. Combination Therapy

In the methods provided herein, the polymorph or mixture of polymorphs may, for example, be employed alone, in combination with one or more other endothelin antagonists, or with another compound or therapies useful for the treatment of diastolic heart failure. For example, the formulations can be administered in combination with other compounds known to modulate the activity of endothelin receptor, such as the compounds described in U.S. Pat. Nos. 6,432,994; 6,683,103; 6,686,382; 6,248,767; 6,852,745; 5,783,705; 5,962,490; 5,594,021; 5,571821; 5,591,761; 5,514,691. Several other endothelin antagonists are described in the literature as described above.

The polymorphs provided herein can be employed in combination with endothelin antagonists known in the art and include, but are not limited to a fermentation product of Streptomyces misakiensis, designated BE-18257B which is a cyclic pentapeptide, cyclo(D-Glu-L-Ala-allo-D-lle-L-Leu-D-Trp); cyclic pentapeptides related to BE-18257B, such as cyclo(D-Asp-Pro-D-Val-Leu-D-Trp) (BQ-123) (see, U.S. Pat. No. 5,114,918 to Ishikawa et al.; see, also, EP A1 0 436 189 to BANYU PHARMACEUTICAL CO., LTD (Oct. 7, 1991)); and other peptide and non-peptidic ETA antagonists have been identified in, for example, U.S. Pat. Nos. 6,432,994; 6,683,103; 6,686,382; 6,248,767; 6,852,745; 5,783,705; 5,962,490; 5,594,021; 5,571821; 5,591,761; 5,514,691; 5,352,800; 5,334,598; 5,352,659; 5,248,807; 5,240,910; 5,198,548; 5,187,195; 5,082,838; 6,953,780; 6,946,481; 6,852,745; 6,835,741; 6,673,824; 6,670,367; and 6,670,362. These include other cyclic pentapeptides, acyltripeptides, hexapeptide analogs, certain anthraquinone derivatives, indanecarboxylic acids, certain N-pyriminylbenzenesulfonamides, certain benzenesulfonamides, and certain naphthalenesulfonamides (Nakajima et al. (1991) J. Antibiot. 44:1348-1356; Miyata et al. (1992) J. Antibiot. 45:74-8; Ishikawa et al. (1992) J .Med. Chem. 35:2139-2142; U.S. Pat. No. 5,114,918 to Ishikawa et al.; EP A1 0 569 193; EP A1 0 558 258; EP A1 0 436 189 to BANYU PHARMACEUTICAL CO., LTD (Oct. 7, 1991); Canadian Patent Application 2,067,288; Canadian Patent Application 2,071,193; U.S. Pat. No. 5,208,243; U.S. Pat. No. 5,270,313; U.S. Pat. No. 5,612,359, U.S. Pat. No. 5,514,696, U.S. Pat. No. 5,378,715; Cody et al. (1993) Med. Chem. Res. 3:154-162; Miyata et al. (1992) J. Antibiot 45:1041-1046; Miyata et al. (1992) J. Antibiot 45:1029-1040, Fujimoto et al. (1992) FEBS Lett. 305:41-44; Oshashi et al. (1002) J. Antibiot 45:1684-1685; EP A1 0 496 452; Clozel et al. (1993) Nature 365:759-761; International Patent Application WO93/08799; Nishikibe et al. (1993) Life Sci. 52:717-724; and Benigni et al. (1993) Kidney Int. 44:440-444). Numerous sulfonamides that are endothelin peptide antagonists are also described in U.S. Pat. Nos. 5,464,853; 5,594,021; 5,591,761; 5,571,821; 5,514,691; 5,464,853; International PCT application No.96/31492; and International PCT application No. WO 97/27979. In certain embodiments, the polymorphs can be administered in combination with sitaxsentan, bosentan or ambrisentan.

Further endothelin antagonists described in the following documents, incorporated herein by reference in their entirety, are exemplary of those contemplated for use in combination with the polymorphs provided herein: U.S. Pat. No. 5,420,123; U.S. Pat. No. 5,965,732; U.S. Pat. No. 6,080,774; U.S. Pat. No. 5,780,473; U.S. Pat. No. 5,543,521; WO 96/06095; WO 95/08550; WO 95/26716; WO 96/11914; WO 95/26360; EP 601386; EP 633259; U.S. Pat. No. 5,292,740; EP 510526; EP 526708; WO 93/25580; WO 93/23404; WO 96/04905; WO 94/21259; GB 2276383; WO 95/03044; EP 617001; WO 95/03295; GB 2275926; WO 95/08989; GB 2266890; EP 496452; WO 94/21590; WO 94/21259; GB 2277446; WO 95/13262; WO 96/12706; WO 94/24084; WO 94/25013; U.S. Pat. No. 5,571,821; WO 95/04534; WO 95/04530; WO 94/02474; WO 94/14434; WO 96/07653; WO 93/08799; WO 95/05376; WO 95/12611; DE 4341663; WO 95/15963; WO 95/15944; EP 658548; EP 555537; WO 95/05374; WO 95/05372; U.S. Pat. No. 5,389,620; EP 628569; JP 6256261; WO 94/03483; EP 552417; WO 93/21219; EP 436189; WO 96/11927; JP 6122625; JP 7330622; WO 96/23773; WO 96/33170; WO 96/15109; WO 96/33190; U.S. Pat. No. 5,541,186; WO 96/19459; WO 96/19455; EP 713875; WO 95/26360; WO 96/20177; JP 7133254; WO 96/08486; WO 96/09818; WO 96/08487; WO 96/04905; EP 733626; WO 96/22978; WO 96/08483; JP 8059635; JP 7316188; WO 95/33748; WO 96/30358; U.S. Pat. No. 5,559,105; WO 95/35107; JP 7258098; U.S. Pat. No. 5,482,960; EP 682016; GB 2295616; WO 95/26957; WO 95/33752; EP 743307; and WO 96/31492; such as the following compounds described in the recited documents: BQ-123 (Ihara, M., et al., “Biological Profiles of Highly Potent Novel Endothelin Antagonists Selective for the ETA Receptor”, Life Sciences, Vol. 50(4), pp. 247-255 (1992)); PD 156707 (Reynolds, E., et al., “Pharmacological Characterization of PD 156707, an Orally Active ETA Receptor Antagonist”, The Journal of Pharmacology and Experimental Therapeutics, Vol. 273(3), pp. 1410-1417 (1995)); L-754,142 (Williams, D. L., et al., “Pharmacology of L-754,142, a Highly Potent, Orally Active, Nonpeptidyl Endothelin Antagonist”, The Journal of Pharmacology and Experimental Therapeutics, Vol. 275(3), pp. 1518-1526 (1995)); SB 209670 (Ohlstein, E. H., et al., “SB 209670, a rationally designed potent nonpeptide endothelin receptor antagonist”, Proc. Natl. Acad. Sci. USA, Vol. 91, pp. 8052-8056 (1994)); SB 217242 (Ohlstein, E. H., et al., “Nonpeptide Endothelin Receptor Antagonists. VI:Pharmacological Characterization of SB 217242, A Potent and Highly Bioavailable Endothelin Receptor Antagonist”, The Journal of Pharmacology and Experimental Therapeutics, Vol. 276(2), pp. 609-615 (1996)); A-1 27722 (Opgenorth, T. J., et al., “Pharmacological Characterization of A-127722: An Orally Active and Highly Potent E.sub.TA -Selective Receptor Antagonist”, The Journal of Pharmacology and Experimental Therapeutics, Vol. 276(2), pp.473-481 (1996)); TAK-044 (Masuda, Y., et al., “Receptor Binding and Antagonist Properties of a Novel Endothelin Receptor Antagonist, TAK-044 {Cyclo [D-α-Aspartyl-3-[(4-Phenylpiperazin-1-yl)Carbonyl]-L-Alanyl-L-α-Aspartyl-D-2-(2-Thienyl)Glycyl-L-Leucyl-D-Tryptophyl]Disodium Salt}, in Human EndothelinA and EndothelinB Receptors”, The Journal of Pharmacology and Experimental Therapeutics, Vol. 279(2), pp. 675-685 (1996)); bosentan (Ro 47-0203, Clozel, M., et al., “Pharmacological Characterization of Bosentan, A New Potent Orally Active Nonpeptide Endothelin Receptor Antagonist”, The Journal of Pharmacology and Experimental Therapeutics, Vol. 270(1), pp. 228-235 (1994)).

The polymorphs provided herein can also be administered in combination with other classes of compounds. Exemplary classes of compounds for combinations herein include endothelin converting enzyme (ECE) inhibitors, such as phosphoramidon; thromboxane receptor antagonists such as ifetroban; potassium channel openers; thrombin inhibitors (e.g., hirudin and the like); growth factor inhibitors such as modulators of PDGF activity; platelet activating factor (PAF) antagonists; anti-platelet agents such as GPIIb/IIIa blockers (e.g., abdximab, eptifibatide, and tirofiban). P2Y(AC) antagonists (e.g., clopidogrel, ticlopidine and CS-747), and aspirin; anticoagulants such as warfarin, low molecular weight heparins such as enoxaparin, Factor VIIa Inhibitors, and Factor Xa Inhibitors, renin inhibitors; angiotensin converting enzyme (ACE) inhibitors such as captopril, zofenopril, fosinopril, ceranapril, alacepril, enalapril, delapril, pentopril, quinapril, ramipril, lisinopril and salts of such compounds; neutral endopeptidase (NEP) inhibitors; vasopepsidase inhibitors (dual NEP-ACE inhibitors) such as omapatrilat and gemopatrilat; HMG CoA reductase Inhibitors such as pravastatin, lovastatin, atorvastatin, simvastatin, NK-104 (a.k.a. itavastatin, or nisvastatin or nisbastatin) and ZD-4522 (also known as rosuvastatin, or atavastatin or visastatin); squalene synthetase inhibitors; fibrates; bile acid sequestrants such as questran; niacin; anti-atherosclerotic agents such as ACAT inhibitors; MTP Inhibitors: calcium channel blockers such as amlodipine besylate; potassium channel activators; alpha-adrenergic agents, beta-adrenergic agents such as carvedilol and metoprolol; antiarrhythmic agents; diuretics, such as chlorothlazide, hydrochiorothiazide, flumethiazide, hydroflumethiazide, bendroflumethiazide, methylchlorothiazide, trichioromethiazide, polythiazide or benzothlazide as well as ethacrynic acid, tricrynafen, chlorthalidone, furosenilde, musolimine, bumetanide, triamterene, amiloride and spironolactone and salts of such compounds; thrombolytic agents such as tissue plasminogen activator (tPA), recombinant tPA, streptokinase, urokinase, prourokinase and anisoylated plasminogen streptokinase activator complex (APSAC); anti-diabetic agents such as biguanides (e.g. metformin), glucosidase inhibitors (e.g., acarbose), insulins, meglitinides (e.g., repaglinide), sulfonylureas (e.g., glimepiride, glyburide, and glipizide), thiozolidinediones (e.g. troglitazone, rosiglitazone and pioglitazone), and PPAR-gamma agonists; mineralocorticoid receptor antagonists such as spironolactone and eplerenone; growth hormone secretagogues; aP2 inhibitors; non-steroidal antiinflammatory drugs (NSAIDS) such as aspirin and ibuprofen; phosphodiesterase inhibitors such as PDE III inhibitors (e.g., cilostazol) and PDE V inhibitors (e.g., sildenafil, tadalafil, vardenafil); protein tyrosine kinase inhibitors; antiinflammatories; antiproliferatives such as methotrexate, FK506 (tacrolimus, Prograf), mycophenolate and mofetil; chemotherapeutic agents; immunosuppressants; anticancer agents and cytotoxic agents (e.g., alkylating agents, such as nitrogen mustards, alkyl sulfonates, nitrosoureas, ethylenimines, and triazenes): antimetabolites such as folate antagonists, purine analogues, and pyrridine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids (e.g., cortisone), estrogens/antiestrogens, androgens/antiandrogens, progestins, and luteinizing hormone-releasing hormone anatagonists, octreotide acetate; microtubule-disruptor agents, such as ecteinascidins or their analogs and derivatives: microtubule-stablizing agents such as pacitaxel (Taxol®), docetaxel (Taxotere®), and epothilones A-F or their analogs or derivatives; plant-derived products, such as vinca alkaloids, epipodophyllotoxins, taxanes; and topoisomerase inhibitors: prenyl-protein transferase inhibitors: and miscellaneous agents such as, hydroxyurea, procarbazine, mitotane, hexamethylmelamine, platinum coordination complexes such as cisplatin, satraplatin, and carboplatin); cyclosporins; steroids such as prednisone or dexamethasone; gold compounds; cytotoxic drugs such as azathiprine and cyclophosphamide: TNF-alpha inhibitors such as tenidap; anti-TNF antibodies or soluble TNF receptor such as etanercept (Enbrel) rapamycin (sirolimus or Rapamune), leflunimide (Arava); and cyclooxygenase-2 (COX-2) inhibitors such as celecoxib (Celebrex) and rofecoxib (Vioxx).

The above other therapeutic agents may be used, for example, in those amounts indicated in the Physicians' Desk Reference (PDR) or as otherwise determined by one of ordinary skill in the art.

The following examples are included for illustrative purposes only and are not intended to limit the scope of the invention.

### EXAMPLE 1

Preparation of Form A

Twenty liters of ethanol were added to five kilograms of N-(2-acetyl-4,6-dimethylphenyl)3-{[(3,4dimethyl-5-isoxazolyl)amino]sulfonyl}-2-thiophenecarboxamide contained in a reactor, heated to 75° C. and stirred until a clear solution was obtained. The solution was filtered and the volume was reduced by approximately 25% while at 75° C. and at atmospheric pressure. The solution was cooled to 45° C. over 30 minutes and held at this temperature for 30 minutes. After the appearance of solids, the solution was cooled to 5° C. over 2 hours and held at this temperature overnight. Filtration of the solution provided a 90% yield of Form A solids.

### EXAMPLE 2

Preparation of Form A

Twenty liters of ethanol were added to five kilograms of N-(2-acetyl-4,6-dimethylphenyl]-3-(((3,4dimethyl-5-isoxazolyl)aminosulfonyl}-2-thiophenecarboxamide contained in a reactor, heated to 75° C. and stirred until a clear solution was obtained. The solution was filtered and the volume was reduced by approximately 25°×6 while at 75° C. and at atmospheric pressure. The solution was cooled to 45° C. over 30 minutes and seed crystals of Form A were added. After the appearance of solids, the solution was cooled to 5° C. over 2 hours and held at this temperature overnight. Filtration of the solution provided a 90% yield of Form A solids.

### EXAMPLE 3

1.0 g of N-(2-acetyl-4,6-dimethylphenyl]-3-(((3,4dimethyl-5-isoxazolyl)aminosulfonyl}-2-thiophenecarboxamide was suspended in 5 mL EtOAc and heated at reflux until a clear solution was obtained. The solution was allowed by cool to room temperature during which off white solids were formed. The solids were collected via filtration, washed with cold EtOAc and dried under vacuum to yield 0.75 g N-(2-acetyl-4,6-dimethylphenyl]-3-(((3,4dimethyl-5-isoxazolyl)aminosulfonyl}-2-thiophenecarboxamide polymorph A.

### EXAMPLE 4

1.0 g N-(2-acetyl-4,6-dimethylphenyl]-3-(((3,4dimethyl-5-isoxazolyl)aminosulfonyl}-2-thiophenecarboxamide was suspended in 10 mL EtOAc and heated at reflux until a clear solution was obtained. While still hot 5 ml hexanes were added and the still clear solution was allowed by cool to room temperature during which off white solids were formed. The solids were collected via filtration, washed with cold EtOAc and dried under vacuum to yield 0.84 g N-(2-acetyl-4,6-dimethylphenyl]-3-(((3,4dimethyl-5-isoxazolyl)aminosulfonyl}-2-thiophenecarboxamide polymorph A.

### EXAMPLE 5

1.0 g N-(2-acetyl-4,6-dimethylphenyl]-3-(((3,4dimethyl-5-isoxazolyl)aminosulfonyl}-2-thiophenecarboxamide was suspended in 10 mL EtOAc and heated at reflux until a clear solution was obtained. While still hot 10 ml hexanes were added and the still clear solution was allowed by cool to room temperature during which off white solids were formed. The solids were collected via filtration, washed with cold EtOAc and dried under vacuum to yield 0.83 g N-(2-acetyl-4,6-dimethylphenyl]-3-(((3,4dimethyl-5-isoxazolyl)aminosulfonyl}-2-thiophenecarboxamide polymorph A.

### EXAMPLE 6

Preparation of Form C

Twenty liters of ethanol were added to five kilograms of N-(2-acetyl-4.6-dimethylphenyl)-3-{((3,4dimethyl-5-isoxazolyl)amino)sulfonyl}-2-thiophenecarboxamide contained in a reactor, heated to 75° C. and stirred until a clear solution was obtained. The solution was filtered and the volume was reduced by approximately 25% while at 75° C. and at atmospheric pressure. The solution was cooled to 45° C. over 30 minutes and held at this temperature for 30 minutes. After the appearance of solids, the solution was cooled to 5° C. over 2 hours and held at this temperature overnight. Filtration of the solution provided a 90% yield of Form C solids.

### EXAMPLE 7

Preparation of Form E

Twenty liters of ethanol were added to five kilograms of N-(2-acetyl-4,6-dimethylphenyl)-3-{((3,4dimethyl-5-isoxazolyl)aminosulfonyl)-2-thiophenecarboxamide contained in a reactor, heated to 75° C. and stirred until a clear solution was obtained. The solution was filtered and the volume was reduced by approximately 25% while at 75° C. and at atmospheric pressure. The solution was cooled from 75° C. to 5° C. over 30 minutes and held at this temperature overnight. Filtration of the solution provided a 90% yield of Form E solids.

### EXAMPLE 8

Preparation of Form E

Twenty liters of ethanol were added to five kilograms of N-(2-acetyl-4,6-dimethylphenyl)-3-{((3,4dimethyl-5-isoxazolyl)aminosulfonyl}-2-thiophenecarboxamide contained in a reactor, heated to 75° C. and stirred until a clear solution was obtained. The solution was filtered and the volume was reduced by approximately 25% while at 75° C. and at atmospheric pressure. The solution was cooled from 75° C. to 45° C. over 30 minutes. Before any solids appeared, a sample of this solution was removed from the reactor and was allowed to rapidly cool at 5° C., solidify which formed seed crystals of Form E. The solution is then cooled to 5° C. and the Form E seed crystals were placed into the reactor. The solution was held at 5° C. overnight. Filtration of the solution provided a 90% yield of Form E solids.

Since modifications will be apparent to those of skill in this art, it is intended that this invention be limited only by the scope of the appended claims.

What is claimed is:

1. A compound N-(2-acetyl-4,6-dimethylphenyl)-3-{((3,4dimethyl-5-isoxazolyl)aminosulfonyl}-2-thiophenecarboxamide, in a form of polymorph C.

2. The compound of claim 1, wherein the amount of polymorph C is more than about 80%.

3. The compound of claim 1, wherein the amount of polymorph C is more than about 85%.

4. The compound of claim 1, wherein the amount of polymorph C is more than about 90%.

5. The compound of claim 1, wherein the amount of polymorph C is more than about 95%.

6. The compound of claim 1, wherein the amount of polymorph C is more than about 98%.

7. The compound of claim 1, wherein the amount of polymorph C is more than about 99%.

8. The compound of claim 1, wherein the amount of polymorph C is about 100%.

9. The compound of claim 10, wherein the polymorph C is characterized by peaks in the XRPD pattern at approximately 7.56, 15.02 and 25.74.

10. The compound of claim 9, wherein the polymorph C is further characterized by peaks in the XRPD pattern at approximately 14.54, 15.96, 16.4, 19.04 and 21.24.

11. The compound of claim 1, wherein the polymorph C is characterized by peaks in the infrared absorption spectra in potassium bromide approximately at 3241 (broad), 1684, 1657, 1525, 1402, 1293, 1140, 1017, 927(broad), 916, 896, 873-, 784, 775, 746-, 728, 706, 680, 653, 580 and 513 cm⁻¹.

12. The compound of claim 1, wherein the polymorph C is characterized by peaks in the Raman absorption spectra approximately at 3083, 2928, 1684, 1654, 1462 and 1291 cm⁻¹.

13. A process for producing Form C as defined in claim 1, comprising the steps of:
dissolving the compound in warmed ethanol to afford a saturated solution; and
slowly cooling the saturated solution to obtain a solid precipitate.

14. The process of claim 13, wherein the ethanol is heated to about 75° C.

15. The process of claim 13, wherein the saturated solution was cooled to about 45° C.

16. The process of claim 15, wherein the saturated solution was further cooled to about 5° C.

17. A compound N-(2-acetyl-4,6-dimethylphenyl)-3-{((3,4dimethyl-5-isoxazolyl)aminosulfonyl}-2-thiophenecarboxamide, in a form of polymorph E.

18. The compound of claim 17, wherein the amount of polymorph E is more than about 80%.

19. The compound of claim 16, wherein the amount of polymorph E is more than about 90%.

20. The compound of claim 17, wherein the amount of polymorph E is more than about 95%.

21. The compound of claim 17, wherein the amount of polymorph E is more than about 98%.

22. The compound of claim 17, wherein the amount of polymorph E is more than about 99%.

23. The compound of claim 17, wherein the amount of polymorph E is about 100%.

24. The compound of claim 17, wherein the polymorph E is characterized by peaks in the XRPD pattern at approximately 10.54, 14.66, 22.44 and 23.82.

25. The compound of claim 24, wherein the polymorph E is further characterized by peaks in the XRPD pattern at approximately 16.2, 20.04, and 24.82.

26. The compound of claim 16, wherein the polymorph E is characterized by peaks in the infrared absorption spectra in potassium bromide are approximately at 3271(broad), 3005, 2982, 1659, 1649, and 1429.

27. The compound of claim 17, wherein the polymorph E is characterized by peaks in the Raman absorption spectra approximately at 3131, 2924, 1659, 1419 and 1304.

28. A process for producing Form E as defined in claim 17, comprising the steps of:
dissolving the compound in warmed ethanol to afford a saturated solution; and
rapidly cooling the saturated solution to obtain a solid precipitate.

29. The process of claim 28, wherein the ethanol is heated to about 75° C.

30. The process of claim 29, wherein the saturated solution was cooled to about 5° C.

31. The process of claim 30, wherein the saturated solution was cooled from about 75° C. to about 5° C. in about 30 minutes.

32. A method for the treatment, prevention or amelioration of an endothelin-mediated disease, comprising administering to a subject an effective amount of the compound of claim 1, wherein the effective amount is sufficient to ameliorate one or more of the symptoms of the disease.

33. The method of claim 32, wherein the disease is selected from the group consisting of hypertension, cardiovascular diseases, cardiac diseases including myocardial infarction, pulmonary hypertension, neonatal pulmonary hypertension, erythropoietin-mediated hypertension, respiratory diseases and inflammatory diseases, including asthma, bronchoconstriction, ophthalmologic diseases including glaucoma and inadequate retinal perfusion, gastroenteric diseases, renal failure, endotoxin shock, menstrual disorders, obstetric conditions, wounds, laminitis, erectile dysfunction, menopause; osteoporosis-and metabolic bone disorders, climacteric disorders including hot flushes, abnormal clotting patterns, urogenital discomfort and increased incidence of cardiovascular disease and other disorders associated with the reduction in ovarian function in middle-aged women, pre-eclampsia, control and management of labor during pregnancy, nitric oxide attenuated disorders, anaphylactic shock, hemorrhagic shock and immunosuppressant-mediated renal vasoconstriction.

34. The method of claim 32, wherein the disease is pulmonary hypertension.

35. A method for inhibiting the binding of an endothelin peptide to an endothelinA (ETA) or endothelinB (ETB) receptor, comprising contacting the receptor with the polymorph of claim 1, or a pharmaceutially acceptable derivative thereof, wherein:
the contacting is effected prior to, simultaneously with or subsequent to contacting the receptor with the endothelin peptide.

36. A method for altering endothelin receptor-mediated activity, comprising contacting an endothelin receptor with the compound of claim 1.

37. A pharmaceutical composition, comprising the compound of claim 1, in a pharmaceutically acceptable carrier.

38. The composition of claim 37 that is formulated for single or multiple dosage administration.

39. An article of manufacture, comprising packaging material and the compound of claim 1, contained within the packaging material, wherein the compound is effective in treating, preventing or ameliorating the symptoms of an endothelin-mediated disorder and the packaging material includes a label that indicates that the compound is used for treating, preventing or ameliorating an endothelin-mediated disorder.

40. A method for the treatment, prevention or amelioration of an endothelin-mediated disease, comprising administering to a subject an effective amount of the compound of claim 17, wherein the effective amount is sufficient to ameliorate one or more of the symptoms of the disease.

41. The method of claim 40, wherein the disease is selected from the group consisting of hypertension, cardiovascular diseases, cardiac diseases including myocardial infarction, pulmonary hypertension, neonatal pulmonary hypertension, erythropoietin-mediated hypertension, respiratory diseases and inflammatory diseases, including asthma, bronchoconstriction, ophthalmologic diseases including glaucoma and inadequate retinal perfusion, gastroenteric diseases, renal failure, endotoxin shock, menstrual disorders, obstetric conditions, wounds, laminitis, erectile dysfunction, menopause; osteoporosis-and metabolic bone disorders, climacteric disorders including hot flushes, abnormal clotting patterns, urogenital discomfort and increased incidence of cardiovascular disease and other disorders associated with the reduction in ovarian function in middle-aged women, pre-eclampsia, control and management of labor during pregnancy, nitric oxide attenuated disorders, anaphylactic shock, hemorrhagic shock and immunosuppressant-mediated renal vasoconstriction.

42. The method of claim 40, wherein the disease is pulmonary hypertension.

43. A method for inhibiting the binding of an endothelin peptide to an endothelina (ET_{A}) or endothelinB (ETB) receptor, comprising contacting the receptor with the polymorph of claim 17, or a pharmaceutially acceptable derivative thereof, wherein:
the contacting is effected prior to, simultaneously with or subsequent to contacting the receptor with the endothelin peptide.

44. A method for altering endothelin receptor-mediated activity, comprising contacting an endothelin receptor with the compound of claim 17.

45. A pharmaceutical composition, comprising the compound of claim 17, in a pharmaceutically acceptable carrier.

46. The composition of claim 45 that is formulated for single or multiple dosage administration.

47. An article of manufacture, comprising packaging material and the compound of claim 17, contained within the packaging material, wherein the compound is effective in treating, preventing or ameliorating the symptoms of an endothelin-mediated disorder and the packaging material includes a label that indicates that the compound is used for treating, preventing or ameliorating an endothelin-mediated disorder.
